(19) United States
(12) Patent Application Publication
(10) Pub. No.: US 2004/0043003 A1
(43) Pub. Date: Mar. 4, 2004

(54) Clinical grade vectors based on natural microflora for use in delivering therapeutic compositions

(76) Inventors: Wei Chen, San Diego, CA (US); Xiaoli Fu, Carlsbad, CA (US); Sherry Nouraini, Vista, CA (US); Zhiqing Zhang, Beijing (CN)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 10/280,769

(22) Filed: Oct. 25, 2002

Related U.S. Application Data
(60) Provisional application No. 60/401,465, filed on Aug. 5, 2002.
(60) Provisional application No. 60/353,885, filed on Jan. 31, 2002.
(60) Provisional application No. 60/353,923, filed on Jan. 31, 2002.
(60) Provisional application No. 60/353,964, filed on Jan. 31, 2002.

(51) Int. Cl. A61K 48/00

(52) U.S. Cl. 424/93.2

(57) ABSTRACT

Clinical grade vectors comprising transformed microflora vector having at least one transforming nucleic acid sequence containing at least one gene of interest are provided. The transforming nucleic acid encodes for a therapeutic polypeptide that is delivered to the host in need thereof topically, orally, intranasally and/or transderamally. The clinical grade vectors are derived from lactic acid bacteria, yeast and other non-pathogenic microorganisms and have been provided with selective/and or reporter genes that do not rely on antibiotic resistance. Moreover, the clinical grade vectors are provided with phenotypic and/or genotypic traits that limit the ex vivo dissemination of transforming nucleic acid sequence(s). Also provided are methods and compositions useful in treating diseases in animals.

### RELATED APPLICATIONS

This application claims priority to provisional application serial No. 60/401465 filed Aug. 5, 2002, No. 60/353885 filed Jan. 31, 2002, No. 60/353923 filed Jan. 31, 2002, and No. 60/353964 filed Jan. 31, 2002 the contents of which are incorporated herein by reference in their entirety.

### FIELD OF THE INVENTION

The present invention relates to treating, palliating or preventing diseases using clinical grade vectors for delivering therapeutic compositions directly to an anatomical site in need thereof. More specifically the clinical grade vectors of the present invention are based on natural microflora and do not possess antibiotic resistance selection markers and have been specifically engineered to limit ex vivo dissemination of transforming nucleic acid sequences.

### BACKGROUND OF THE INVENTION

The last twenty-five years have brought tremendous scientific advances in the fields of molecular biology and genetics. Our understanding of gene function, heterologous gene expression and the underling molecular basis for many diseases has grown proportionately with these scientific advances. However, many of the most promising and exciting medical applications of molecular biology have yet to be realized. There are many technical and ethical challenges that must be overcome before new molecular based therapeutics and prophylactics become a viable option for health care providers and recipients.

Generally, gene-based clinical applications presently being developed include vaccines, gene-replacement therapies and therapeutic composition delivery. Host cell transformation can be accomplished using gene-delivery vectors comprising replication incompetent viruses (see for example U.S. Pat. No. 5,824,544), naked DNA, (see for example U.S. Pat. No. 6,261,834), liposome containing recombinant expression cassettes (see for example U.S. Pat. No. 6,271,207), and microflora vectors (see for example U.S. provisional patent application serial Nos. 60/353,885 and 60/353,923). Gene-delivery vectors that secrete and/or surface express the therapeutic compostions are described in U.S. provisional patent application serial Nos. 60/353,885 and 60/353,923). Other molecular-based therapeutic composition delivery approaches include using replication incompetent recombinant viruses designed to express a heterologous surface proteins (see for example U.S. Pat. No. 6,376,236).

Recombinant therapeutic compositions are presently prepared in vitro. Large scale bioreactors are used to grow massive quantities of therapeutic composition secreting cells and the recombinant therapeutic composition-rich supernatant is harvested and concentrated. The therapeutic composition is then extracted, purified and compounded using classical pharmacological techniques. This process is extreme costly and often results in poor yields and denatured proteins. Consequently, pharmaceutical researchers have attempted to develop methods for in vivo therapeutic composition expression using recombinant organism-based vectors, inanimate vectors and naked DNA.

Examples of recombinant organism-based vectors include recombinant bacteria (see for example U.S. Pat. No. 5,547,664) and viruses such as alphaviruses (see for example U.S. Pat. No. 6,391,632), vaccinia viruses (see for example U.S. Pat. No. 6,267,965), adenoviruses (see for example U.S. Pat. No. 5,698,202) and adenovirus associated virus (AAV) (see for example U.S. Pat. No. 6,171,597). Inanimate vectors include lipidic gene delivery vector constructs such as DNA/cationic liposome complexes, DNA encapsulated in neutral or anionic liposomes, and liposome-entrapped, polycation-condensed DNA (LPDI and LPDII). (Ropert, C. 1999. Liposomes as a gene delivery system. Braz J Med Biol Res;32(2):163-9.) However, many technical difficulties remain to be overcome before the bacterial and viral vectors can be used in vivo gene delivery. Moreover, no successful human clinical trials using lipid vectors have been conducted to date.

Much of what is known about gene delivery vectors came for pioneering work in the filed of gene therapy. Gene therapy is a term coined to describe three distinct therapeutic models. The most common form of gene therapy is gene replacement therapy whereby a host cell (target cell) previously incapable of providing a necessary gene product is transformed into a gene product producing cell. In most cases inherited disorders such as cystic fibrosis, severe combined immunodeficiency syndrome (SCID) and ornithine transcarbamylase deficiency (OTCD) have been the gene replacement research focus. Another gene therapy approach involves the in vivo synthesis of a gene product wherein the transgene product is itself a therapeutic agent or palliative. For example, a vector encoding for a cytotoxic agent is administered to a cancer patient. The vector transforms the cancer cell (target cell) and the resulting transgene expression kills the cancer cell. The third model is similar to the second. However, instead of encoding for a therapeutic agent, the transgene encodes a gene sequence that activates or augments existing apoptotic mechanisms within the target cell; again, transgene expression results in cell death.

Target cell transformation can be accomplished either ex vivo or in vivo depending on the target cell, the nature of the transgene, and the transgene product. Cells transformed ex vivo are re-introduced into the host following transformation resulting in in vivo gene expression (see for example U.S. Pat. No. 5,399,346). In vivo transformation requires that the transgene containing vector itself be administered as a therapeutic (see for example U.S. Pat. No. 6,015,694). Regardless of how the cell is transformed, gene therapy consists of contacting a target cell with a gene delivery vector having a nucleic acid construct encoding a therapeutic transgene. In the case of inherited disorders such as those previously described, the therapeutic transgene replaces a missing or defective host gene. Consequently, the host is provided with a transformed cell population that produces a gene product that the recipient's natural cells do not.

There are significant differences between in vivo and ex vivo gene delivery. When a host cell is transformed ex vivo the vector is not administered directly to the host. Therefore, there is less risk of vector-associated adverse reactions than when the vector is administered directly. For example, in one of gene therapy's successes, W French Anderson and colleagues transformed SCID patients' T lymphocytes with a gene encoding the enzyme adenosine deaminase (ADA) ex vivo. The results were so successful that ADA transformation gene therapy has been approved (outside the US only) as a SCID therapy. (Anderson W F, Blaese R M, Culver K. 1990. The ADA human gene therapy clinical protocol: Points to consider response with clinical protocol, Jul. 6, 1990. Hum Gene Ther Fall;1(3):331-62; see also Blaese R M, Culver K W, Chang L, Anderson W F, Mullen C, Nienhuis A, Carter C, Dunbar C, Leitman S, Berger M, et al. 1993. Treatment of severe combined immunodeficiency disease (SCID) due to adenosine deaminase deficiency with CD34+ selected autologous peripheral blood cells transduced with a human ADA gene. Amendment to clinical research project, Project 90-C-195, Jan. 10, 1992. Hum Gene Ther Aug;4(4):521-7).

Unfortunately, not all inherited diseases can be effectively treated using ex vivo transformation. Moreover, much like in vitro recombinant therapeutic composition production, ex vivo cell transformation is a difficult and time consuming enterprise that is not easily adapted to wide scale use. Consequently, in vivo gene delivery methods for gene therapy as well as vaccines is an active and dynamic research area.

Initially, in vivo gene delivery centered on the use of recombinant viral vectors. However, the theoretical dangers associated with using viral vectors for gene therapy and vaccines became apparent when University of Pennsylvania researchers tested an in vivo gene replacement therapy for patents suffering from partial OTCD. (See Batshaw M L, Wilson J M, Raper S, Yudkoff M, Robinson M B. 1999. Recombinant adenovirus gene transfer in adults with partial ornithine transcarbamylase deficiency (OTCD). Hospital of the Univ. of Pennsylvania General Clinical Research Center, Philadelphia 19104, USA. Hum Gene Ther Sep 20;10(14):2419-37.) In one experiment an 18 year old OTCD patient was administered a recombinant adenovirus transforming vector containing the ornithine transcarbamylase transgene. The vector was presumed replication incompetent and therefore safe for human administration. Unfortunately, four days after intravenous administration of a recombinant adenovirus vector containing the ornithine transcarbamylase transgene, the patient developed a massive systemic inflammatory immune response and died.

However, severe systemic inflammatory responses are just one of the many safety concerns associated with viral-based, infectious vectors. Other risks include induction of secondary malignancies, recombination to form replication-competent virus and vector directed systemic immune responses that reduce or eliminate the vector's efficacy on subsequent administration. Therefore, researchers are investigating nonviral transgene vectors. Although existing non-viral vectors are generally not as efficient as viral vectors, nonviral systems have the potential advantage of being less toxic, nonrestrictive in transgene size, potentially targetable, and easy to produce in relatively large amounts. More importantly, nonviral vectors generally lack immunogenicity, allowing repeated in vivo transfection using the same vector.

Recently, a new approach to in vivo gene delivery and therapeutics has been developed that uses vectors derived from recombinant natural microflora (see for example the present inventors U.S. provisional patent application serial Nos. 60/353,885 and 60/353,923). Vectors derived from organisms such as, but not limited to Lactobacillus sp., Lactococcus sp, Steptococcus sp., Saccharomyces sp and others have been developed. These are particularly desirable because natural microflora vectors are largely immunologically inert, non-pathogenic, well characterized and are present in foods and therefore generally regarded as safe (GRAS) by regulatory agencies. Moreover, when used to deliver immunogenic antigens, expressed on their surfaces or secreted, recombinant natural micro-flora vectors deliver their antigen payloads directly to immunocompetent cells such as intestinal M cells.

Furthermore, while not as desirable as natural microflora, other researchers are developing bacterial vectors derived from human pathogens including Shigella sp. Salmonella sp. and Listeria monocytogenes (see for example U.S. Pat. Nos. 6,287,556, 6,210,663 and 6,004,815). However, vectors derived from potentially pathogenic agents, while attractive due to their natural cell invasiveness, present health risks during manufacturing, distribution and use. Consequently, it is unlikely that vectors derived from pathogens will be widely used in human medicine.

However, regardless of whether GRAS organisms such as microflora or attenuated pathogens such as enteric organisms are used for therapeutic applications, the vectors themselves must be safe and easily adapted to changing manufacturing environments. One of the most difficult challenges will be developing bacterial vectors without using selection markers based on antimicrobial resistance. Briefly, whenever a bacterial population is transfected with a transgene containing plasmid only a portion of the original population is successfully transformed. Consequently, it is necessary to be able to identify the transformed bacteria from the non-transformed ones. This requires a selection marker, an identification marker, or both. For clinical grade vectors it is desirable to only use one, preferably a selection marker. The selection marker most commonly used in molecular biology is an antibiotic resistance gene. The antibiotic resistance gene is fused to the plasmid nucleic acid generally downstream of the gene of interest and driven by the same promoter. However, numerous variations on this scheme are possible. After the transformation step the bacterial population is plated on a culture medium containing a bactericidal or bacteristatic concentration of antibiotic. Bacteria successfully transformed will express the antibiotic resistance gene and replicate; untransformed bacterial will not. Consequently, the transformed bacterial having the therapeutic gene of interest will be easily identified and subsequently purified.

Transgene vectors selected in this fashion will also have an antibiotic resistance gene either present in an extrachromosomal plasmid, or integrated into its genome. In either event there is a significant risk that the antibiotic resistance marker will be transmitted to other organisms in the host or the environment. Furthermore, if attenuated enteric pathogens are used as vectors as proposed in the cited U.S. patents above, pathogenic reversion coupled with antibiotic resistance present an unacceptable public health threat. Therefore, there remains a need for non-pathogenic, immunologically inert transgene vectors capable of high in vivo expression that can be directed to specific target cells without the risk of transferring antibiotic resistance markers to unintended hosts.

### SUMMARY OF THE INVENTION

The present invention provides recombinant vectors for delivering therapeutic compositions directly to anatomical sites in need thereof. The clinical grade vectors of the present invention are derived from natural microflora that have been adapted to secrete and/or surface express therapeutic compositions. The delivery vectors made in accordance with the teachings of the present invention are composed of live non-pathogenic yeast or bacteria expressing and secreting a therapeutic protein. The non-pathogenic yeast or bacterial vectors of the present invention have a distinct advantage over other non-pathogenic yeast or bacterial vectors vector systems previously described. Specifically, the non-pathogenic yeast or bacterial vectors of the present invention do not use antibiotics for selection of bacteria and/or yeast recombinants.

Moreover, another advantage of the therapeutic protein delivery vectors made in accordance with the teachings of the present invention is that dissemination of unwanted genes to the environment or resident microfloral yeast and bacteria is avoided. The present inventors also provide methods and compostions for targeting the bacterial and yeast vectors to the epithelial layer of the gut and other mucosal membranes.

In one embodiment of the present invention vector selection is accomplished using microflora having one or more housekeeping gene either deleted from its genome or rendered inoperable. Consequently, absent an operable replacement gene the microflora organism cannot survive and/or replicate.

In another embodiment of the present invention the operable replacement gene is provided on a plasmid operably linked to a gene of interest and driven by the same promoter. Microflora vectors of the present invention transformed with a plasmid having both the gene of interest and replacement housekeeping gene thrive while non-transformants fail to proliferate.

In one embodiment of the present invention microflora are vectors are selected having a mutation in a critical replication enzyme such as, but not limited to thymidylate synthase (thyA).

In another embodiment of the present invention clinical grade vectors are provided with reporter genes expressed from a constitutive promoter cloned into the expression vector and used as a screening tool. Non-limiting examples of reporter genes suitable for use in accordance with the teachings of the present invention include green fluorescent protein (GFP), β-galactosidase, amylase, and chloramphenicol acetyl transferase (CAT).

The vectors described above, and made in accordance with the teachings of the present invention have been termed “Clinical Grade Vectors.” As used herein the term “clinical grade” refers to vectors that do not possess antibiotic resistance genes or use resistance to antibiotics as a method for section. Furthermore, “clinical grade vectors” may also include variants designed to have limited, or no, survival capability outside the host. The host being defined herein as an intended recipient of the clinical grade vectors of the present invention. In this context it should be noted that many microorganisms including the natural strains of microflora organisms used to prepare the clinical grade vectors of the present invention may be naturally resistant to one or more antibiotic. The antibiotic resistance that the microflora organisms naturally exhibit, regardless of its mechanism or genetic organ, is not considered an “antibiotic resistance gene” as used herein. The term “antibiotic resistance gene” as used by the present inventors refers to antibiotic resistance purposely conferred on the natural organism as a means of selecting transformed organisms. It is understood that many of the microorganisms used as clinical grade vectors of the present invention may possess naturally occurring antibiotic resistance genes.

In another embodiment of the present invention the clinical grade vectors are used to deliver a heterologous gene of interest to a host. The gene of interest may encode for therapeutic compositions and transgenes, including, but not limited to hormones (such as, but not limited to alpha-melanocyte-stimulating hormone (α-MSH), insulin, growth hormone, and parathyroid hormone) and cytokines (including, but not limited to: interferons, interleukin (IL)-2 interleuki-4, interleukin-10, interleukin-12, G-CSF, GM-CSF, and EPO).

In still another embodiment of the present invention methods for treating or palliating an inflammatory disease in an animal are provided.

In another embodiment of the present invention the inflammatory disease is uveitis and the animal is selected from the non-limiting group consisting of primates, equine, bovine, porcine, ovine, rodents, fish, and birds.

In one embodiment of the present invention the method of treating or palliating an inflammatory disease is a method of treating or palliating uveitis by the administration of a clinical grade vectors of the present invention expressing alpha-melanocyte-stimulating hormone ((α-MSH) to a host in need thereof.

### BRIEF DESCRIPTION OF THE DRAWINGS

FIGS. 1 and 2 depict the selection of thyA⁻ mutants in accordance with the teachings of the present invention.

FIG. 3 depicts construction of the gram-positive expression vector pSYMX.

FIG. 4 depicts Saccharomyces cerevisiae expression vector p426GPD made in accordance with the teachings of the present invention.

FIG. 5 depicts a flow chart showing the steps involved in construction of a secreted αMSH expression vector made in accordance with the teachings of the present invention.

FIG. 6 schematically depicts the construction of yeast cell-wall display vector pGPD-dsply made in accordance with the teachings of the present invention.

FIGS. 7A and 7B schematically depicts integration of expression vectors of the present invention into the yeast genome.

FIG. 8 schematically depicts plasmid pSYM6 made in accordance with the teachings of the present invention.

FIG. 9 schematically depicts plasmid pSYM3 made in accordance with the teachings of the present invention.

FIG. 10 diagrammatically depicts construction of a Thy A deletion strain and reporter gene (GFP) made in accordance with the teachings of the present invention.

### DEFINITION OF TERMS

Prior to setting forth the invention, it may be helpful to an understanding thereof to set forth definitions of certain terms that will be used hereinafter.

An “antibiotic resistance gene” as defined herein includes heterologous nucleic acid sequences purposely provided to a vector and used as a selection system. The term “antibiotic resistance gene” does not include other mechanisms or genes that impart antibiotic resistance to naturally occurring micro-flora organisms.

“Clinical grade vector” as used herein means a therapeutic compound and/or gene delivery vector comprising a non-pathogenic bacteria or yeast derived from the natural microflora. The clinical grade vectors of the present invention do not use antibiotic resistance markers for selection and/or have been modified to prevent replication outside the host.

“Detectable immune response” as used herein is either an antibody (humoral) or cytotoxic (cellular) response formed in an animal in response to an antigen that can be measured using routine laboratory methods including, but not limited to enzyme-linked immunosorbant assays (ELISA), radio-immune assays (RIA), immunofluorescent assays (IFA), complement fixation assays (CF), Western Blot (WB) or an equivalent thereto.

“Gene of interest” as used herein refers to any nucleic acid sequence encoding for a polypeptide or protein whose expression is desired. The gene of interest may or may not include the promoter or other regulatory components.

“Gene therapy” as used herein is defined as the delivery of a gene of interest to an animal in need thereof using a recombinant vector. The gene of interest can be a transgene encoding for a therapeutic or prophylactic protein or polypeptide including, but not limited to cytokines, anti-inflammatories, anti-proliferatives, antibiotics, metabolic inhibitors/activators and immunologically active antigens and fragments thereof. Furthermore, “gene therapy” as used herein also includes gene replacement technologies directed at both inherited and non-inherited disorders.

“Host” as used herein defined the intended recipient of a therapeutic composition of the present invention. Host includes all animals. Specifically, hosts include, but are not limited to, primates (including man), bovine, equine, canine, feline, porcine, ovine, rabbits, rodents, birds and fish.

A “housekeeping gene” as used herein refers to a nucleic acid sequence found in the host genome or extrachomosomal DNA that is expressed following interaction between a promoter and RNA polymerase without out additional regulation (constitutive expression). The housekeeping genes of the present invention are essential for the cell's activity. Mutations therein, or a complete deletion of the gene, renders the cell incapable of growth absent supplementation or genetic augmentation (e.g.: transforming the cell having the defective or missing housekeeping gene with an operable one).

“Immunologically inert” as used herein shall mean any substance, including microorganisms such as microflora, that do not provoke a significant immune response in its host. Examples of immunologically inert materials as used herein include stainless steel, biocompatible polymers such as poly-L-lactide, medical grade plastics and the microflora organisms of the present invention. A “significant immune” response is any immune response that would limit or restrict the in vivo utility of a material or organism used in accordance with the teachings of the present invention. A detectable immune response is not necessarily a “significant immune response.”

An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example, (a) a DNA molecule which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such a cDNA or genomic DNA library.

“Microflora” as used herein refers to non-pathogenic bacteria and yeast naturally associated with the human body. Non-limiting examples include lactic acid bacteria and yeast. Microflora bacteria and yeast generally do not provoke an immune response in the host or recipient and are ubiquitous in all species of animals.

“Percent identity (homology)” of two amino acid sequences or of two nucleic acids is determined using the algorithm of Karlin and Altschul (Proc. Natl. Acad. Sci. US 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide (e.g., SEQ ID NO: 2). To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See http://www.ncbi.nim.nih.gov.

“Reporter gene” as used herein is a nucleic acid sequence incorporated into the heterologous nucleic acid encoding for the gene of interest that provided the transformed vector expressing the gene of interest an identifiable phenotype. Non-limiting examples of reporter genes include green fluorescent Protein (GFP), β-galactosidase, amylase, and chloramphenicol acetyl transferase (CAT).

“Screening marker” as used herein refers to an identifying characteristic (phenotype) provided to a transformed vector made in accordance with the teachings of the present invention. In one embodiment of the present invention the screening marker is a reporter gene.

“Selectable marker,” “selectable gene,” “reporter gene” and “reporter marker” (referred to hereinafter as a “selectable marker”) as used herein refer to nucleic acid sequences encoding for phenotypic traits that permit the rapid identification and isolation of a transformed bacterial vector. Generally, bacterial vectors deemed “clinical grade” and made in accordance with the teachings of the present invention are those vectors having selectable markers that do not encode for antibiotic resistance.

“Transgene” as used herein refers to a gene that is inserted, using cDNA technology, into a cell in a manner that ensures its function, replication and transmission as a normal gene.

“Transforming nucleic acid sequence” as used herein means a plasmid, or other expression cassette containing a nucleic acid sequence encoding a gene of interest. In some embodiments of the present invention the nucleic acid sequence can encode for one or more gene products encoded for by a defective or missing housing keeping gene. In another embodiment of the present invention the transforming nucleic acid sequence may encode for both a gene of interest and a housekeeping gene. “Transforming nucleic acid sequence” can also be used to mean a “transgene” in accordance with certain embodiments of the present invention. In another embodiment of the present invention the transforming nucleic acid sequence includes nucleic acid sequence encoding for a promoter and/or other regulatory elements.

### DETAILED DESCRIPTION OF THE INVENTION

I. Introduction

Delivery of therapeutic compositions and nucleic acids to specific target sites within the human body is an ongoing challenge faced by the drug development industry. The present inventors have developed clinical grade vectors composed of non-pathogenic microorganisms having therapeutic peptides and/or proteins expressed on their surface, or secreted therefrom. In another embodiment of the present invention the non-pathogenic microorganisms include, but are not limited to, gram positive lactic acid bacteria (LAB) such as Lactobacillus acidophilus, Lactobacillus brevis, Lactobacillus casei, Lactobacillus plantarum, Lactobacillus delbrueckii, Lactobacillus delbrueckii subsp. Bulgaricus, Lactobacillus helveticus, Lactobacillus pentosus, Lactobacillus fermentum, Lactobacillus amylovorus, Lactococcus lactic, Lactococcus cremoris, Streptococcus, Streptococcus gordonii, gram negative bacteria such as Escherichia coli and Caulobacter crescentus, and the common baker's yeast Saccharomyces cerevisiae.

The present invention therefor comprises new compositions and methods for their preparation and use. In one embodiment of the present invention the clinical grade vectors are made using species of microflora selected from the non-limiting group consisting of lactic acid bacteria, Escherichia coli, Caulobacter crescentus and Saccharomyces cerevisiae. The clinical grade vectors can be transformed with genes encoding for proteins or polypeptides having therapeutic value. The transformed clinical grade vectors of the present invention can be used topically or systemically to treat or palliate disease.

In one embodiment of the present invention a therapeutic protein such as, but not limited to anti-inflammatory agent, neuropeptides and interleukins is provided to a site in need thereof. In an exemplary non-limiting example the present invention is used to deliver recombinant alpha-melanocyte-stimulating hormone (rα-MSH) to the eye as a treatment for uveitis.

When used in accordance with the teachings of the present invention LAB are particularly interesting delivery vector candidates for therapeutic compositions or their respective nucleic acids. Briefly, LAB are indigenous microflora of mammalian gastrointestinal tract that play an important role in the host microecology and have been credited with an impressive list of therapeutic properties. These therapeutic properties include, but are not limited to the maintenance of microbial ecology of the gut, physiological, immuno-modulatory and antimicrobial effects. Other LAB associated attributes include enzyme release into the intestinal lumen that act synergistically with LAB adhesion to alleviate symptoms of intestinal malabsorption. Furthermore, the LAB enzymes help regulate intestinal pH which results in increased aromatic amino acid degradation. [Fuller, R. Probiotic foods—current use and future developments. IFI NR 3:23-26 (1993); Mitsuoka, T. Taxonomy and ecology of Bifidobacteria. Bifidobacteria Microflora 3:11 (1984); Gibson, G. R. et al., Probiotics and intestinal infections, p.10-39. In R. Fuller (ed.), Probiotics 2: Applications and practical aspects. Chapman and Hall, London, U.K. (1997); Naidu A S, et al., Probiotic spectra of lactic acid bacteria (LAB). Crit Rev Food Sci Nutr 39:3-126 (1999); Naidu, A. S., Clemens, R. A. Probiotics, p.431-462. In A. S. Naidu (ed.), Natural Food Antimicrobial Systems. CRC Press, Boca Raton, Fla. (2000)]. Lactic acid bacteria of the present invention include, but are not limed to the following genera: Streptococcus, Enterococcus, Lactococcus, Lactobacillus, Leuconostoc, Pediococcus and Bifidobacteria.

Microflora generally have many qualities that are desirable in recombinant vectors intended for in vivo use. Microflora are non-pathogenic, are immunologically inert, and are well characterized, both phenotypically and. genetically. Microflora are generally not fastidious and multiply rapidly thus they are readily adaptable to large scale manufacturing. Moreover, microflora are common in food and natural products and are generally regarded as safe (GRAS) for human consumption by regulatory agencies. However the present generation of bacterial-based vectors, including LAB, have used antibiotic resistance genes as selectable rendering them unsuitable for in vivo use. The present invention provides alternatives to using antibiotic resistance genes as methods for identifying transformed micro-flora.

Selectable markers provide researchers and technicians a convenient means for distinguishing transformed microorganisms from non-transformed ones in a mixed population. One of the oldest, and most effective means of identifying transformed organism is to incorporate a selectable marker nucleic acid sequence into the plasmid containing the gene of interest. The selectable marker sequence is generally inserted downstream of the gene of interest and is driven off the same promoter. As a result, cells successfully transformed with the gene of interest will also be transformed with selectable marker nucleic acid sequence. When antibiotic resistance is used as the selectable marker, only transformed cells will survive and/or grow in media containing the antibiotic.

Thus, antibiotic resistance is a convenient and much used phenotype when developing transformants. However, vectors having antibiotics resistant genes as selective markers are capable of horizontal gene transfer that can endow other organisms with antibiotics-resistant phenotypes. This risk is especially acute when microflora organisms such as LAB are used for therapeutic vectors. Therefore, regulatory agencies do not allow the use of antibiotic-resistant markers in live attenuated vaccine strains.

In order to use microflora as a gene delivery system to humans, the present inventors have developed a clinical grade vector system that does not use an antibiotic selection marker. One of the alternatives to using antibiotic resistance genes provided by the present invention includes clinical grade vectors having chromosomal deletions or lethal mutations in an essential house-keeping gene. Next, a functional analogous house-keeping gene is inserted into a plasmid encoding for a gene of interest. Consequently, the house-keeping gene becomes the selectable marker allowing for the rapid identification and isolation of transformants. The essential house-keeping gene can encode for any number of metabolic regulators and/or enzymes including, but not limited to kinases, proteases, synthetases, dehydrogenases and others. Another alternative to antibiotic resistance genes provided by the present invention includes clinical grade vectors having reported genes incorporated into the plasmid containing the gene of interest. Non-limiting examples of reporter genes used in accordance with the teachings of the present invention include green fluorescent Protein (GFP), β-galactosidase, amylase, and chloramphenicol acetyl transferase (CAT).

In other embodiments of the present invention the analogous housekeeping gene and gene of interest are contained in an integrated expression cassette that is incorporated into the host genomic DNA rather than as an extrachromasomal plasmid.

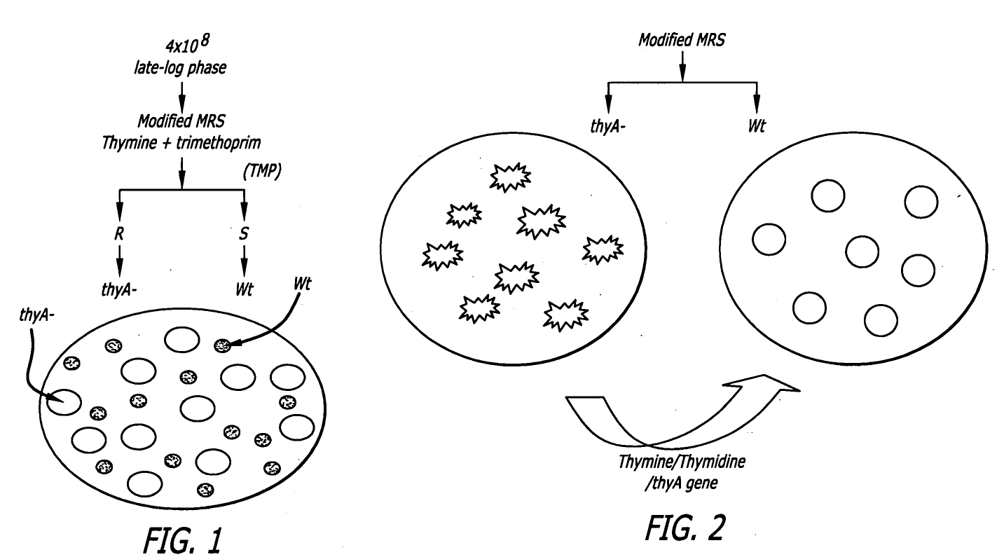

In one embodiment of the present invention a clinical grade vector comprising the gene encoding for thymidylate synthase (thyA) is selected. ThyA is required for DNA synthesis. It catalyzes the conversion of dUMP and 5,10-methylenetetrahydrofolate to dTMP and 7,8-dihydrofolate. ThyA mutant strains are unable to grow in media lacking or having limited amounts of thymidine or thymine. Therefore, the present inventors selected for thyA mutant strains of Lactobacilli in order to develop the clinical grade selection system of the present invention. Screening bacterial populations for specific deletion mutants is well known in the art and well within the skill of the ordinary bacteriologist or molecular biologist. Briefly, thyA mutants are identified based on resistance to trimethoprim, which blocks conversion of 5,10-methylenetetrahydrofolate to 7,8-dihydrofolate by thyA gene product as described in: Fu, X and J G Xu. 2000. Development of a Chromosome-Plasmid Balanced Lethal System for Lactobacillus acidophilus with thyA Gene as Selective Marker. Microbiol. Immunol. 44(7), 551-556. To select for trimethoprim-resistant mutants (thyA mutant) LAB are grown in a modified MRS medium containing 20 μg/ml trimethoprim and 50 μg/ml thymine (FIG. 1). Then, Trimethoprim-resistant mutants are transformed with a plasmid containing the thyA gene. Transformants are selected on modified MRS medium in the absence of thymine (FIG. 2).

Integrated expression cassettes used in accordance with the teachings of the present invention can be integrated into yeast and bacterial chromosomes. For example, and not intended as a limitation, heterologous genes are commonly expressed in yeast on episomal plasmids, whose stable maintenance inside the cell requires continuous selective pressure by regulating the composition of the growth medium. Although the composition of the growth medium can be tightly controlled in vitro, in vitro nutrient regulation is generally not possible. Consequently, loss of the expression plasmid from yeast cells over time, and reduce the efficacy of the yeast protein delivery system is probable. To circumvent this potential problem, the present inventors have devised methods for integrating expression cassettes into the yeast chromosome by homologous recombination. Chromosomal integration endows expression cassettes with stable maintenance, and removes the requirement for a specially designed growth medium. An added and extremely important advantage of chromosomal integration is prevention of horizontal transfer of the DNA molecule to other yeast strains or species.

Two methods are provided to integrate expression cassettes into the yeast chromosome. In one embodiment an integrative vector is provided that lacks sequences required for replication in yeast, but contains sequences homologous to a specific chromosomal locus. Once transformed into yeast, the integrative vector enters the chromosome by homologous recombination with the homologous chromosomal site (See FIG. 7A). However, it is possible that the target chromosomal sequence can be duplicated upon the expression cassette's chromosomal integration, (See FIG. 7A). lntrachromosomal homologous recombination between these duplicated sequences can reverse the chromosomal integration of the expression cassettes and lead to expression cassette.

Therefore, the present inventors have provided an alternative method whereby expression cassette loss is minimized. In this embodiment of the present invention, the expression cassette is integrated as a PCR product in the absence of a plasmid as shown in FIG. 7B. In this embodiment, chromosomal integration is mediated by sequences at the ends of the PCR product that have homologies to flanking sequences of a target site on the chromosome. Consequently, this method provided homologous recombination leading to gene-replacement, as opposed to duplication, of the target sequence making the chromosomal integration irreversible (FIG. 7B).

The preceding non-limiting examples are applicable to any otherwise clinically acceptable vector including naked plasmid DNA or integration expression cassettes. However, for purposes of the following non-limiting exemplary embodiment of the present invention four different organisms will be used. These will be gram-negative bacteria E. coli and Caulobacter crescentus, gram-positive lactic acid bacterium Lactococcus lactis, and baker's yeast Saccharomyces cerevisiae. Expression in Saccharomyces cerevisiae will be on episomal vectors, as well as on integrative vectors introduced into the yeast chromosome. The vectors used for each expression system were either purchased from commercial sources or prepared using methods known to those having ordinary skill in the art. Details for each expression system are outlined in Table 1 below.

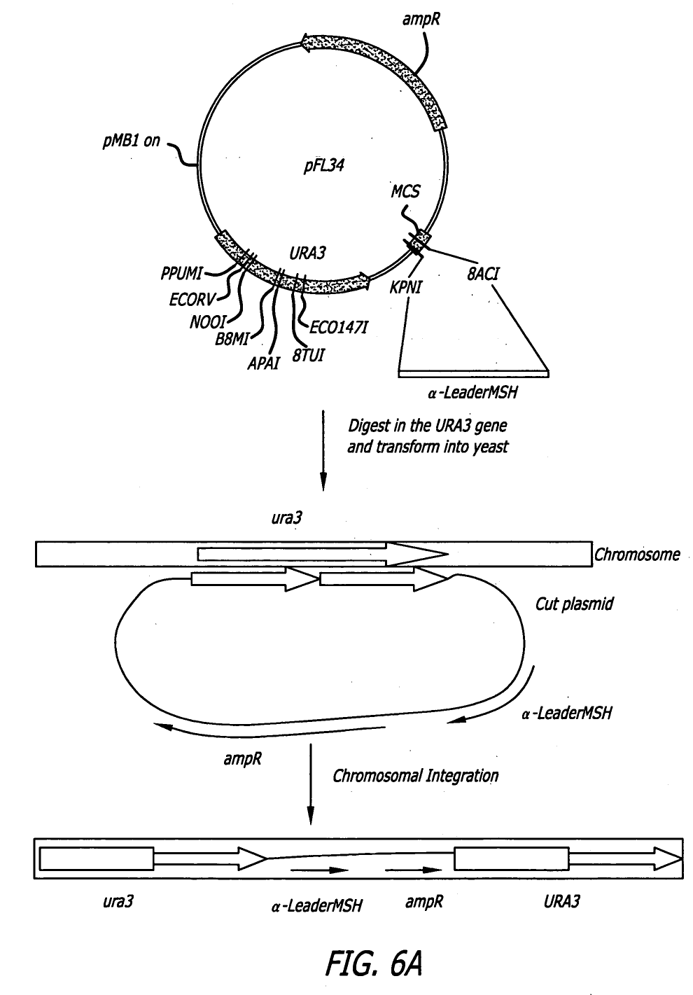

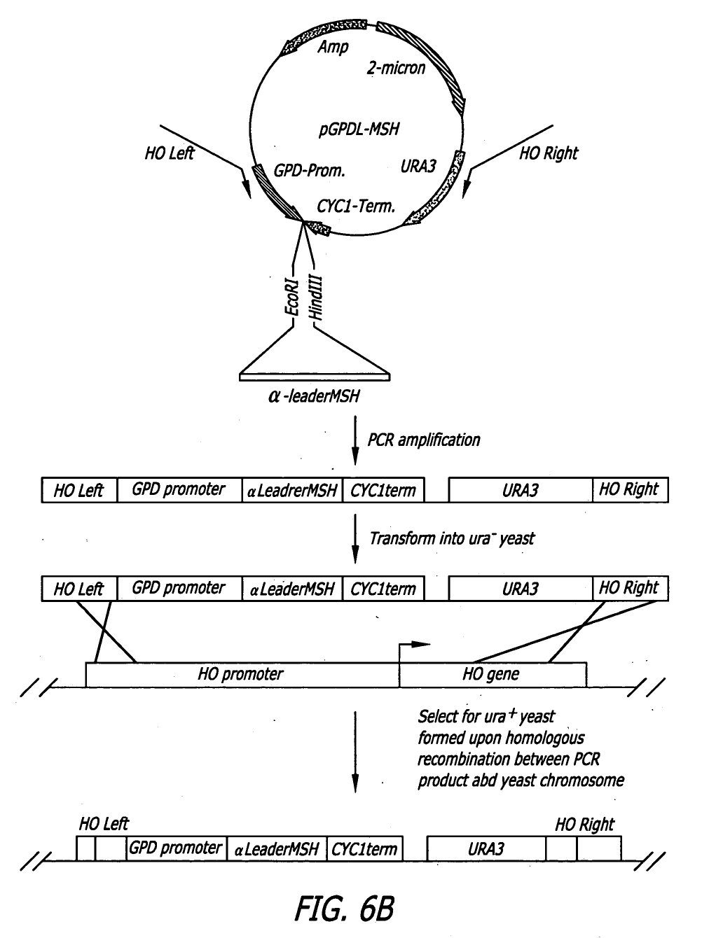

TABLE 1
Exemplary Vectors of the Present Invention

| | Target | Mode of | | Reference or |
|---|---|---|---|---|
| Plasmid | organism | expression | Promoter | Source |
| pCX | Caulobacter | Secretion | Plac/Inducible | Invitrogen¹ |
| | crescentus | | | |
| pFliTrx | E. coli | Display on | λphage | Lu² et al., |
| | | surface of | P_{L}/inducible | 1995 |
| | | flagella | | |
| pSYMB | LAB | Secretion | P32/constitutive | Symbigene |
| pYD1 | Saccharomyces | Display | Gal1 from S. | Invitrogen¹ |
| | cerevisiae | on cell | cerevisiae | |
| | | wall | /Inducible | |
| p426GPD | Saccharomyces | Intra- | GPD from S. | Mumberg³ et |
| | cerevisiae | cellular | cerevisiae/ | al, 1995 |
| | | | Constitutive | |
| pGPD- | Saccharomyces | Display | As p426GPD | Symbigene |
| DSPLY | cerevisiae | on cell | | (See FIG. 6) |
| | | wall | | |
| pSecY | Saccharomyces | Secretion | As p426GPD | Symbigene |
| | cerevisiae | | | (See FIG. 6) |
| pFL34 | Saccharomyces | None | None | ATCC |
| | cerevisiae | | | |
| pHY304 | Lactobacillus | None | None | Yim⁴ et al., |
| | | | | 1998 |

¹Invitrogen Life Technologies 1600 Faraday Avenue, Carlsbad, CA 92008
²Lu, Z. et al. (1995) Bio/Technology 13:366-372
³Mumberg D. et al. (1995) Gene 156:119-122
⁴Yim et al. (1998) Methods Cell Sci. 20:13-20.

The non-limiting exemplary expression vectors and plasmids in Table 1 are tested for clinical efficacy using the disease models as described below. Both secretion and surface expression models are tested. As previously discussed, the clinical grade vectors of the present invention can be used to deliver genes of interest that encode for therapeutic compositions and transgenes including, but not limited to cytokines and hormones (including, but not limited to: interferons, interleukin-2, interleukin-4, interleukin-12, G-CSF, GM-CSF, EPO, insulin, growth hormone, and parathyroid hormone).

Another aspect of the clinical grade vectors of the present invention includes providing vectors that minimize or eliminate transgene dissemination into the environment or to resident microflora including bacteria and yeast. Uncontrolled spread of the expression of therapeutic proteins could lead to unwanted side-effects. Therefore, the present inventors have conceived of methods and compostions useful in preventing dissemination of expression vectors beyond the host.

In one embodiment the expression cassette is integrated into a locus essential for cell (vector's) growth. For example, the ThyA locus could be disrupted by integration thus generating a thymidine requirement for the vector organism. In the absence of supplied thymidine the vectors will have a limited life span in the host.

In another embodiment cell-death induction is controlled in the microorganism. This may be done by engineering the delivery microorganisms to carry an inducible toxin gene. Once the microorganisms have been introduced into the host, and upon delivery of the therapeutic proteins to the target site, the cells can be eliminated by induction of the expression of the toxic gene. An example of such an inducible system is the elegant, light activated promoter described by Peter Quail at UC Berkeley (Shimizu-Sato S., Huq E., Tepperman J. M., and Quail P. H. (2002), Nature Biotech. 20: 1041-1044). The light activation of this promoter system is dependent on the presence of a non-toxic chromophore. Expression of the toxic gene can be induced at will by providing the chromophore; upon discharge of the microflora vehicles along with intestinal waste, exposure to light activates the toxic gene and eliminates the recombinant microorganisms. Other inducible promoters that may be used with the clinical grade vectors of the present invention include, but are not limited to, a pH inducible promoter as described in U.S. Pat. No. 6,242,194 issued to Kullen , et al., a lactose inducible promoter such as that used in E. Coli plasmids (e.g. pBluescript from Stratagene) or the endogenous lactose promoter in lactobacillus; promoters induced during anaerobic growth such as the promoter for alcohol dehydrogenase (adhE), as described in Aristarkhov, A. et al., “Translation of the adhE Transcript to Produce Ethanol Dehydrogenase Requires Rnase III Cleavage in Escherichia coli,” J. Bacteriology, Vol. 178, No. 14, 4327-4332.

Non-limiting examples of toxic genes include bacterial autolysins under the control of an inducible promoter. The autolysing gene may then be triggered at the appropriate time and place in the gastrointestinal tract through the use of one or more of the inducible promoters described immediately above. Examples of autolysing gene include, but not limited to, AcmA (Buist G., et al., (1997) “Autolysis of Lactococcus lactis caused by induced overproduction of its major autolysin, AcmA,” Appl. Environ. Microbiol, 63:27222728); holin and lysin (Henrich, B., et al., (1995) “Primary Structure and Functional Analysis of Lysis Genes of Lactobacillus gasseri Bacteriophage ˜adh,” J. Bacteriology, Vol. 177, No. 3, 723732). Examples of yeast toxic genes are the killer toxin SMK1 (Suzuki C et al., Lethal effect of the expression of a killer gene SMK1 in Saccharomyces cerevisiae. Protein Eng 2000,13:73-6). In addition, over expression of mammalian pro-apoptotic Bcl-2 family protein Bax results in yeast cell death (Zha et al., Structure-function comparisons of the proapoptotic protein Bax in yeast and mammalian cells. Mol Cell Biol 1996,16:6494-508).

Engineering microorganisms that are sensitive to oxygen is another method for limiting dissemination of the clinical grade vectors of the present invention. The environment of the human gut is very low in oxygen, suitable for growth of anaerobic microorganisms, including the bacterial organisms described in this disclosure. Thus, an efficient means of eliminating microflora delivery vehicles once they have exited the human body upon discharge of intestinal waste into the oxygen-rich outside environment, is to engineer genes into the delivery microorganisms that confer oxygen sensitivity

Moreover, the clinical grade vectors of the present invention may be lysed through infection with bacteriophages following their administration. Non-limiting examples of suitable bacteriophages include φadh, φLC3, mv4, M13, T4, φ29, Cp-1, Cp-7, and Cp-9. The bacteriophages may be introduced hours or days after the first ingestion of engineered bacteria. The first ingestion of the bacterial culture is allowed to colonize the intestines and multiply in number. A second bacterial culture infected with the bacteriophage is then administered hours or days after the first culture. When the bacteriophage lyse the cells in the intestine, phage particles may further infect and lyse the engineered bacteria, thus preventing the dissemination of the genetic material to the outside environment.

Exemplary embodiments of the present invention include, but are not limited to the following therapeutic proteins

1. Alpha melanocyte stimulating hormone (α-MSH)

α-MSH is a neuropeptide, acting as a messenger molecule among the nervous, endocrine and immune systems. This molecule may be important to central and peripheral events that control fever, the acute phase response, immunity and inflammation.

2. Interleukin-10 (IL-10)

Interleukin-10 (IL-10) was first recognized as a “cytokine synthesis inhibitory factor” (CSIF), which is produced by Th2 cells, and inhibits the production of interferon γ (IFNγ), interleukin-2 (IL-2) and tumor necrosis factor beta (TNFβ) by Th1 cells. IL-10 can also inhibit the production of inflammatory cytokines such as IL-1α, IL-1β, IL-6 and TNFα, as well as chemokines such as IL-8. Due to these anti-inflammatory activities, supplementation of IL-10 could potentially be used as a therapy in the treatment of inflammatory and autoimmune diseases. Our delivery system may be used to deliver IL-10 into the body. Human IL-10 is composed of 160 amino acids, with a molecular weight of 18.5 KD. Human IL-10 has a significant degree of sequence homology with bovine, murine, and ovine IL-10, but species specificity exists, since human IL-10 does not bind to murine IL-10 receptor. For animal test of the IL-10 activity, we will clone and express the mouse IL-10 cDNA.

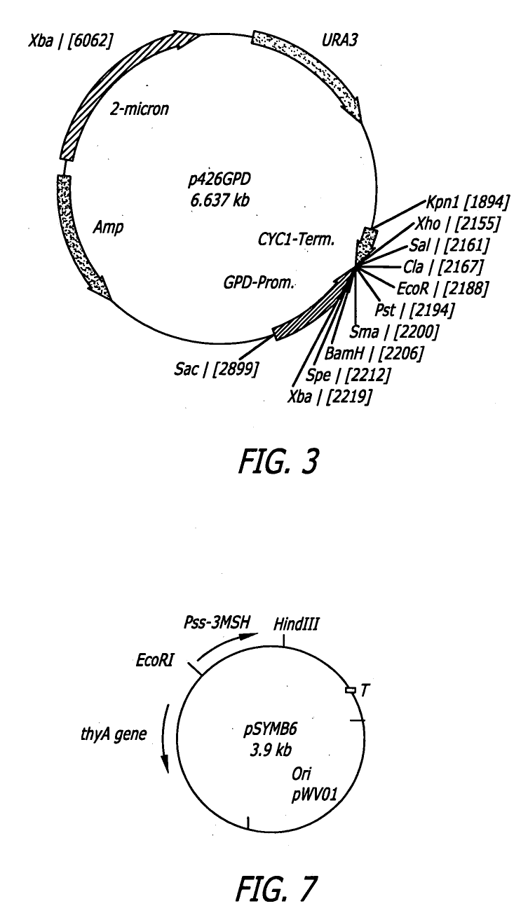

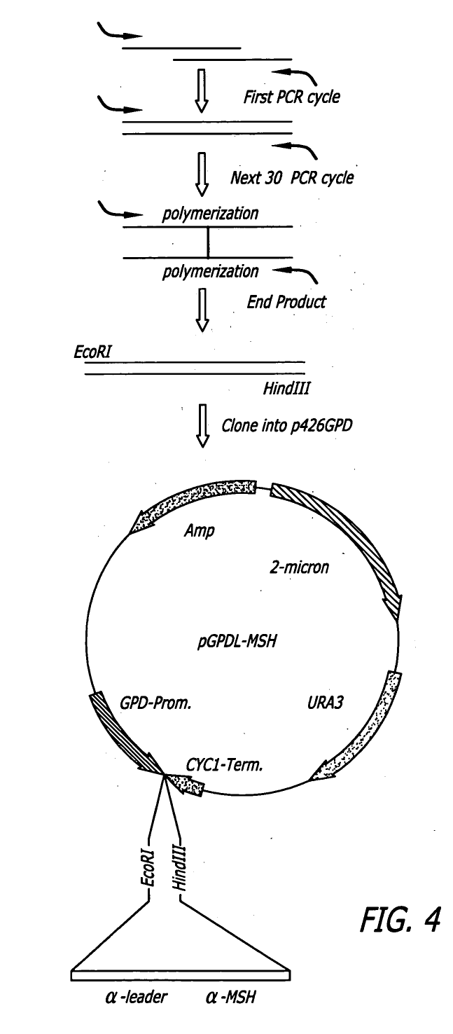

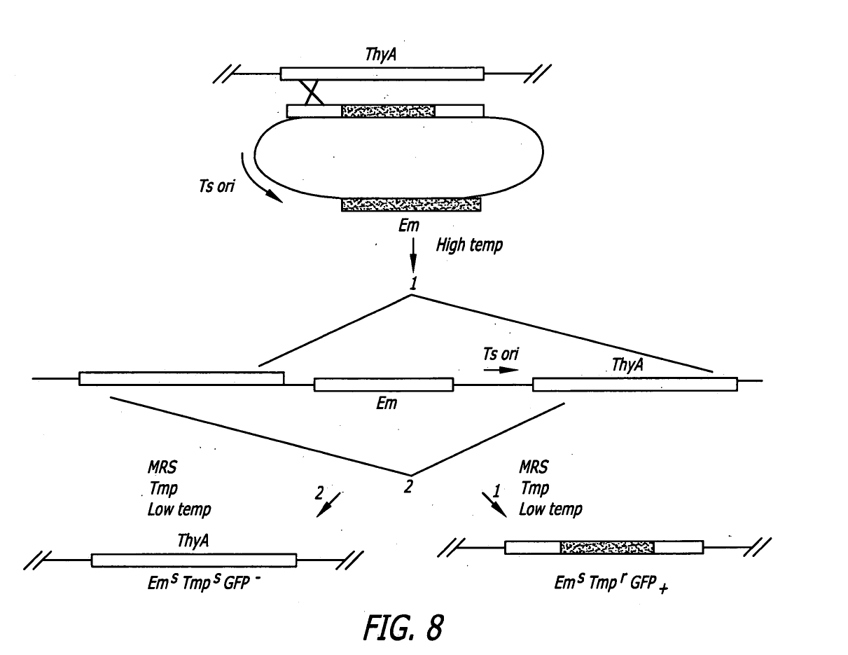

TABLE 2
Plasmid Constructs of the Present Invention

| Plasmid Designation | Gene of Interest |
|---|---|
| pCX-MSH | MSH |
| pCX-3MSH | MSH |
| pSYMB3 | MSH (FIG. 3) |
| pSYMB4 | MSH see |
| pSYMX-MSH | MSH |
| pSYMX-IL-10 | IL-10 |
| pGPDL-MSH, pInt-MSH | MSH |
| pLongα-MSH, pInt-Longα-MSH | MSH |
| pLongα-sp-MSH, pInt-Longα-sp- | MSH |
| MSH | |
| pGPDL-IL10, pInt-IL10 | IL-10 |
| pSYMB6 | ThyA, (FIG. 8) |
| pSYMB7 | ThyA integration |
| pSYMB8 | ThyA integration |

### EXAMPLES

The invention is illustrated by the following Examples. These Examples are presented for illustrative purposes only and are not intended to limit the invention.

### Example 1

### Bacterial and Yeast Strains and Their Growth Media

Bacterial and yeast strains and their growth media: E. coli K12 strain Top10F′ (Invitrogen) was used for cloning. For expression purposes the following strains were used: E. coli strain GI826 (for pFliTrx-based vectors), Caulobacter JS4000, Lactobacillus casei, Lb. plantarum, Lb. brevis, Lb. acidophilus and lactococcus lactis and other subspecies, Saccharomyces cerevisiae strains W303-1a (for p426GPD-based expression vectors) and Eby100 (Invitrogen, for pYD1-based expression vectors). Yeast were either grown on YPD or selective drop-out media, both purchased from Qbiogene. E. coli were grown on LB or RM media (for pFLiTrx system) supplemented with Ampicilin (50-100 μg/ml) or Chloramphenicol (2-15 μg/ml). Caulobacter cells were grown in M11 medium (Invitrogen) supplemented with Chlroamphenicol. Lactobacilli were grown in MRS medium supplemented with Erythromycin (2-50 μg/ml) and Lactococcus on M17 with 0.1% glucose and Erythromycin (2-50 μg/ml). Yeast were transformed according to the protocol described in http://www.umanitoba.ca/faculties/medicine/biochem/gietz/method.html. Bacterial cells were transformed by the method described by Raya et al. (1992, J. Bact. 174 (17):5584-5592).

### Examples 2-14

### Plasmid Construction

All restriction enzymes and DNA modifying enzymes were purchased from New England Biolabs. Hotstart pfu and Hotstart Herculase polymerases were from Stratagene. Oligonucelotides were purchased from Genset Oligos, and sequencing was performed by Qiagen sequencing services. All constructs described below were verified by sequencing. All plasmid DNA preparations were done using a miniprep kit from Qiagen.

### Example 2

### Preparation of Plasmid pCX-MSH

To express MSH in the pCX expression vector, two complementary oligonucleotides were synthesized which contained the following components:

Bgl II recognition site at 5′-end and Not I recognition site at the 3′-end.

coding sequences for human α-MSH, which has been modified to contain codons optimized for expression in prokaryotic cells and in yeast.

Linker sequences composed of poly-Glycine and Serine or Alanine which ensure conformational stability to α-MSH in the context of a fusion protein.

The sequence for one of the two oligonucleotides is shown: (SEQ ID NO: 1) MSHUP SEQ ID NO 3 (5′-AGA TCTGGT GGC GGT GGC TCT TAT TCT ATG GAA CAT TTT CGT TGG GGT AAA CCT GTT GGT GGC GGT GCG GCC GCG-3′). Equimolar amounts of the two MSH-encoding oligonucleotides were mixed and annealed. Modified pCX was linerized with restriction enzyme Bgl II and Not I, ligated with the annealed α-MSH gene fragment that was digested with the same enzymes and transformed into E. coli.

### Example 3

A double stranded oligo encoding three tandem copies of MSH-encoding sequences, separated by 5 or 6 amino acids, were synthesized and cloned into plasmid PCRBlunt (Invitrogen) by Retrogen Inc to botain PCRBluntMSH. The top strand of the oligo is shown below in SEQ. ID NO: 2

```
Arg Ser Leu Asp Gly Gly Gly Gly Ser Tyr Ser Met Glu His Phe Arg
AGA TCT CTA GAT GGT GGC GGT GGC TCT TAT TCT ATG GAA CAT TTT CGT
    Bgl II Xba I
Trp Gly Lys Pro Val Gly Gly Gly Ala Ala Ala Ser Tyr Ser Met Glu
TGG GGT AAA CCT GTT GGT GGC GGT GGC GCG GCC GCG TCT TAT TCT ATG GAA
                                                                                Not I
His Phe Arg Trp Gly Lys Pro Val Gly Gly Gly Gly Gly Ser Tyr Ser
CAT TTT CGT TGG GGT AAA CCT GTT GGT GGT GGC GGT GGC TCT TAT TCT
Met Glu His Phe Arg Trp Gly Lys Pro Val Gly Glu Leu Glu
ATG GAA CAT TTT CGT TGG GGT AAA CCT GTT GGT GAG CTC GAG
                                                                                        Sac I Xhol
TAA GGA TCC
          Bam HI
```

pCX-3MSH was constructed by isolation of 3MSH sequences on a BgIII/Xhol fragment and subcloning into similarly digested pCX vector (Invitrogen).

### Example 4

### Preparation of Plasmid pSYM3

Plasmid pSYM3 contains an MSH secretion cassette composed of the promoter of lactate dehydrogenase gene of Lb. casei (Pldh), the secretion signal from amylaseA gene of Lb. amylovorus (-ss), and sequences encoding three tandem repeats of MSH. This plasmid was constructed as follows: First, a 252 bps of double stranded oligonucleotide (P-ss), formed by hybridizing two complementary single-stranded oligos, was cloned into pCR-Blunt vector. The resulting plasmid is pCR-BluntVP-ss. P-ss contains the LDH promoter followed by the secretion signal and the first 10 codons for the amylase A gene. EcoRI and KpnI sites were designed into 5′ and 3′ ends, respectively, of the Pss fragment. The sequence of Pss top strand appears below (SEQ ID NO: 3. The EcoRI and KpnI sites are underlined).

5′-GAATTCTGAAAAAGTCTGTCAATTTTGTTTCGGCGAATTGATAATGT
GTTATACTCACAATGAAATGCAGTTTGCATGCACATAAGAAAGGATGATA
TCACCGTGAAAAAAAAGAAAAGTTTCTGGCTTGTTTCTTTTTTAGTTATA
GTAGCTAGTGTTTTCTTTATATCTTTTGGATTTAGCAATCATTCTAAACA
AGTTGCTCAAGCGGCTAGCGATACG ACATCAACTGATCACTCAAGCAAT
GGTACC-3′

Next, the Pss fragment was subcloned into plasmid pUC19 by using the EcoRI/KpnI sites. The resulting plasmid was named pSYMB1. To obtain MSH sequences, PCRBluntMSH was used as a template to amplify triple-MSH using oligos Tri-MSH. For (5′-GGGGTACCAGATCTCTAGATGGTGGC-3′, [SEQ ID NO: 4]. Underlined is a KpnI recognition site) and Tri-MSHRev (5′-CCCAAGCTTGGATCCTTACTCGAGCTCACC-3′, [SEQ ID NO: 5]. Underlined is a HindIII recognition site). The resulting PCR product was digested with KpnI/HindIII and cloned into the same sites of pSYMB1 in frame with the amyA secretion sequences to obtain pSYMB2. Finally, to obtain pSYMB3, the MSH secretion cassette was isolated from pSYMB2 on an EcoRI/HindIII fragment and cloned into the shuttle vector PSYMB that was similarly cut.

### Example 5

### Preparation of Plasmid pSYMB4

To construct pSYMB4, the PCR amplified triple MSH sequences (see above) were cloned into the XbaI/HindIII sites of pSYMB.

### Example 6

### Preparation of Plasmid pSYMX

Plasmid pSYMX is a shuttle vector that can replicate in both gram-positive and gram-negative bacteria. The various components of this vector were assembled on plasmid pBC SK (+) (Stratagene); these components are listed below:

The erythromycin resistant gene (Em) from Staphylococcus aureus plasmid pE194 (ATCC)-The gram-positive origin of replication from pWV01 (ATCC)

The thyA gene of Lb. casei which provides a clinical grade selection system in a thyA mutant host

The transcription termination sequence of the splA gene from Lb. brevis (ATCC 8287) (tslp)

The promoter of lactate dehydrogenase gene of Lb. casei (Pldh) and the secretion signal from amylaseA gene of Lb. amylovorus (-ss)

The various components described above were assembled on pBC SK(+) as follows (see also FIG. 3): the Em gene was isolated from pE 194 on a ClaI/HpaII fragment and cloned into pBC SK(+) that was cut with ClaI, to obtain pBCE. As a result of this construction, pBCE will have only one ClaI site, since hybridization and subsequent ligation of ClaI and HpaII DNA ends destroys the recognition sites for both enzymes.

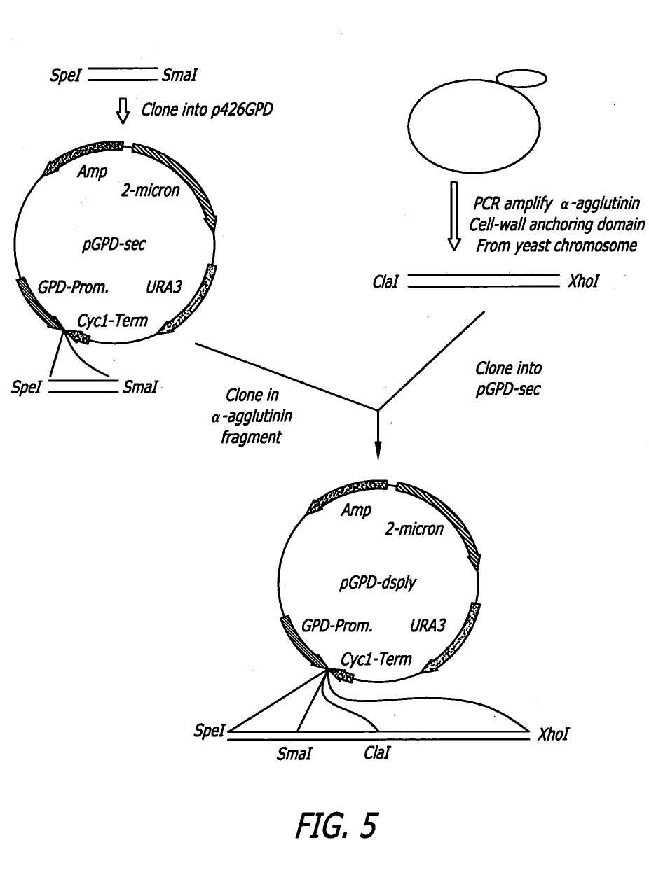

Next, the origin of replication from pWV01 was isolated on a ClaI fragment and cloned into pBCE that was similarly cut. To include the thyA gene in the construction, it was PCR amplified from Lb. casei chromosome with primers thyAPstlFor SEQ ID NO 6: (5′-AACTGCAGTGCAGGCACAGCTTGATGCG-3′) and thyAHindIIIRev SEQ ID NO 7: (5′-cccaagc ttCCTTTTGTGTCATTGGTAAACC-3′), digested with Pstl and HindIII and cloned into similarly cut pBCEW to obtain pBCEWT. tslp was constructed by an overlapping PCR strategy (see preparation of plasmid pgpdl-msh below and FIG. 5 for a general description of this strategy) using tslpABamHI/Pstlup SEQ ID NO 8: (5′-TGATAATTATTATTTAGG TGAGCTTTGTTGATAAAAAGGTCTTTTCMCGTTTATGTTGGGGAGACC-3′) tslpABamHI/PstIIow SEQ ID NO 9: (5′GTTTTTCCTAACAAAGGCCTMITTTTTTCMTATAAAAAGGT CTCCCCMCATAAACGTTGAAAAGACC-3′) as long primers and tslpABamHIFor SEQ ID NO 10: (5′-CGGGATCCTGATAATTATTATTTAGGTG-3′) andtslpAPstIRev SEQ ID NO 11: (5′-AACTGCAGGTTT TTCCTAACAAAGGCC-3′) as outside PCR primers. The final tslp PCR product was digested with BamH I and Pst I and cloned into similarly cut PBCEWT to obtain pBCEWTt.

To obtain the lactate dehydrogenase (LDH) promoter of L. casei (ATCC 393), and the secretion signal of amylase (amyA) from L. amylovorus, a 252 bps of double stranded oligonucleotide (P-ss), formed by hybridizing two complementary single-stranded oligos, was cloned into pCR-Blunt vector. The resulting plasmid is pCR-Blunt/P-ss. P-ss contains the LDH promoter followed by the secretion signal and the first 10 codons for the amylase A gene . The sequence of P-ss top strand appears below:

SEQ ID NO 12

5′-GAATTCTGAAAAAGTCTGTCAATTTTGTTTCGGCGAATTGATAATGT
GTTATACTCACAATGAAATGCAGTTTGCATGCACATAAGAAAGGATGATA
TCACCGTGAAAAAAAGAAAAGTTTCTGGCTTGTTTCTTTTTTAGTTATAG
TAGCTAGTGTTTTCTTTATATCTTTTGGATTTAGCAATCATTCTAAACAA
GTTGCTCAAGCGGCTAGCGATACGACATCAACTGATCACTCAAGCAATGG
TACC-3′:

Finally, to complete construction of pSYMX, the P-ss secretion signal was PCR amplified from pCR-Blunt/P-ss with oligonucleotides P-ssSacIIFor SEQ ID NO 13: (5′-TCCCCGCGGTGAAAAAGTCTGTCMTTTTG-3′) and P-ssXbaIRev SEQ ID NO 14: (5′-GCTCTAGAA TTGCTTGAGTGATCAGTTG-3′), digested with SacII and XbaI and cloned into similarly cut pBCEWTt.

### Example 7

### Preparation of Plsamid pSYMX-MSH

Two oligonucleotides, MSH/XBAI/BAMHIUP SEQ ID NO 15: (CTAGATCTTATTCTATGGAACATTTT CGTTGGGGTAAACCTGTTTAATGAG′-3′) and, MSH/XBAI/BAMHILOW SEQ ID NO 16: (5′-GATCC TCATTAAACAGGTTTACCCCAACGAAAATGTTCCAGAGAATAAGAT-3′) were hybridized to form a double stranded DNA molecule encoding MSH with compatible ends for cloning into the expression vector PSYMX. this double-stranded DNA molecule was cloned into the XBAI/BAMHI sites of pSYMX to obtain pSYMX-MSH.

### Example 8

### Preparartion of Plasmid pSYMX-IL-10

The cDNA for mouse IL-10 was PCR amplified from a mouse lymphocyte cDNA library (Clontech), using primers IL-10XbaIFor SEQ ID NO 17: (5′-TCATCTAGAAAAGCAGGGGCCAGTAC AGC-3′), and IL-10BamHIRev SEQ ID NO 18: (5′-CCCGGATCCTTAGCTTTTCATTTTGATC-3′), the IL-10 PCR product was digested with XbaI and BamHI and ligated to pSYMX-MSH vector that was similarly cut. The resulting plasmid is pSYMX-IL-10 in which a fusion gene encodes IL-10 fused N-terminally to the amylase a sequences. In addition to the secretion signals and promoters used for the gram-positive constructs of the present invention, the present inventors also use vector constructs having promoter and secretion signals of slpA-surface layer protein as of Lactobacillus brevis ATCC 8287 (American Type Culture Collection, Manassas, Va.) and/or secretion signal of usp45 encoding a secreted protein from lactococcus lactis subspecies lactis mg 1363.

### Example 9

### Preparation of Yeast MSH Expressing Vectors

Three different MSH producing plasmids were constructed. pGPDL-MSH contains 20 amino acids from the leader sequence of secreted yeast α-mating factor fused to MSH encoding sequences. In plasmids pLongoαMSH and pLongoα-sp-MSH, the sequences from the α-mating factor were extended to 85 amino acids to include the recognition site for Kex2 protease, which removes the α-leader sequences from MSH. In contrast, pGPDL-MSH derives expression of MSH that is secreted as a fusion to the first 20 amino acids of α-mating factor. The MSH sequence is separated by a two-amino acid spacer from α-leader sequences in constructs pGPDL-MSH and pLongα-sp-MSH; whereas, in pLongα-MSH, the α-leader peptide is directly fused to MSH.

PGPDL-MSH was constructed as follows: A fusion of α-leader-α-MSH was constructed by an overlapping PCR strategy (FIG. 5). Two long oligonucleotides, ALPHALEADER (SEQ ID NO: 19) and MSHPEPTIDE (SEQ ID NO: 20), were synthesized which are complementary at their 3′-ends. ALPHALEADER (SEQ ID NO: 19) (ATG AGA TTT CCT TCA ATT TTT ACT GCA GTT TTA TTC GCA GCA TCC TCC GCA TTA GCT GCT GGT GCT TCT TAC TCT ATG) encodes the α-leader peptide followed by two amino acid spacers and the first four amino acids of α-MSH. MSHPEPTIDE (SEQ ID NO: 20) (TTA AAC TGG CTT ACC CCA TCT GAA GTG TTC CAT AGA GTA AGA AGC ACC AGC AGC TAA TGC) comprises the non-coding strand of the MSH gene followed by sequences complementary to the 3′-end of the ALPHALEADER oligonucleotide. During a PCR reaction, the above mentioned long oligos hybridize and form a template for pfu polymerase to construct a double stranded molecule encoding an α-leader-α-MSH fusion protein. In the same PCR tube, two additional PCR amplification oligonucleotides were included which amplify this fusion construct, and at the same time provide restriction enzyme recognition sites used for cloning. The PCR oligonucleotides used were, PCR fwd (SEQ ID NO: 21) (GGGAATTCATGAGATTTCCTTCAATTTTTAC), and PCR rev (SEQ ID NO: 22) (GGAAGCTTTTAAACTGGCTTACCCC). The final PCR product, digested with EcoRI and HindIII, was cloned into p426GPD, to obtain pGPDL-MSH.

pLongα-sp-MSH and pLongαMSH plasmids were constructed as follows: the first 85 amino acids of the α-mating factor was PCR amplified from yeast chromosome using oligos LALPHAfwd (SEQ ID NO: 23) (5′-ATGAGAUTTTCCTTCAATTTT TACTGC-3′) and either LALPHArev (SEQ ID NO: 24) (5′-ATAGAGTAAGAAGCACCTCT TTTATCCAAAGATACCC-3′) or LALPHAw/osprev (SEQ ID NO: 25) (TGTTCCATAGAGTAAGA TCTTTTATCCAAAGATACCC), to generate PCR products A and B, respectively. In the sequence of reverse oligos, the underlined sequences correspond to the reverse strand of the 3′-end of MSH, and nucleotides shown as bold refer to the reverse strand of spacer-amino acid codons. PCR products A and B were used as templates for subsequent PCR reactions to construct Longα-sp-MSH and LongαMSH fusions, respectively. The latter PCR reactions were primed with oligos EcoLALPHAfwd (SEQ ID NO: 26) (5′-GCGAATTCATGAGATTTCCTTCAATTTTTAC-3′) in combination with either primer LALPHAMSHrev (SEQ ID NO: 27) (5′GGAAGCTTAAACTGGCT TACCCCATCTGAAGTGTTCCATAGAGTAAGAAGCACCTC-3′) or LALPHAMSHw/oSPrev (SEQ ID NO: 28) (5′ GGMGCTTAAACTGGCTTACCCCATCTGAAGTG TTCCATAGAGT) for construction of Longα-sp-MSH or LongαMSH, respectively. The final PCR products were cloned into the EcoRI/HindIII sites of p426GPD to construct MSH expression plasmids pLongα-MSH and pLongα-sp-MSH.

### Example 10

### Construction of GFP-cell Wall Display Vector

pGPD-DSPLY functions as a target vector for expression of proteins displayed on the cell wall. Names and sequences of PCR primers used to construct pGPD-DSPLY and it's derivatives are listed in Table 3. pGPD-DSPLY contains sequences encoding the leader sequence of yeast α-mating factor and the cell-wall anchoring domain (C-terminal 350 amino acids) of Saccharomycse cerevisiae α-agglutinin. First, sequences encoding the a-leader peptide followed by two amino acid spacers (Gly and Ala) were PCR amplified from the yeast chromosome (strain S288C) using primers BamLALPHAfwd and EcoLALPHArev and cloned into BamHI and EcoRI sites of p426GPD to construct pSecY. Next, sequences encoding the cell-wall anchoring domain of α-agglutinin was PCR amplified from yeast chromosomal DNA (strain S288C), using the oligonucleotides Agglfwd and Agglrev, and cloned into the ClaI/Xhol sites of p426GPD to obtain pGPDAnch. pGPD-DSPLY was constructed by subcloning an EcoRI/Xhol fragment containing α-agglutinin sequences into the same sites of pSecY.

The vector for surface display of GFP was constructed as follows: GFP encoding sequences were PCR amplified from plasmid pQB125-fPA (Qbiogene) using primers sgGFPfwd and sgGFPrev and cloned upstream of α-agglutinin sequences into the EcoRI/HindIII sites of pGPDAnch to obtain pGFPAnch. Next, an EcoRI/Xhol fragment from pGFPAnch was subcloned into the same sites of pSecY to obtain pGFPDSPLY.

TABLE 3

SEQ ID NO used for construction of surface
display expression vectors

| | | |
|---|---|---|
| SEQ ID NO | Oligonucleotide | Sequence |

| | | |
|---|---|---|
| SEQ ID NO: 29 | BamLALPHAfwd | 5′-CCGGATCCATGAGATTTCCTTCAATTTTTAC-3′ |
| SEQ ID NO: 30 | EcoLALPHArev | 5′-GCGAATTCAGCACCTCTTTTATCCAAAGATACC-3′ |
| SEQ ID NO: 31 | Agglfwd | 5′-CCATCGATGGTTC TGCTAGCGCCAAAAGCTC-3′ |
| SEQ ID NO: 32 | Agglrev | 5′-CAGCTCGAGTTAGAATAGCAGGTACGAC-3′ |
| SEQ ID NO: 33 | sgGFPfwd | 5′-CGGAATTCATGGCTAGCAAAGGAGAAG-3′ |
| SEQ ID NO: 34 | sgGFPrev | 5′-GGAAGCTTTTAATCGATGTTGTACAGTTC-3′ |

### Example 11

### Preparation of Plasmid pGPD-IL-10

Sequences encoding mature mouse IL-10 protein were PCR amplified from a mouse cDNA library and cloned into the SmaI/HindIII sites of pSecY. The following ligos were used for the PCR reaction: IL-10fwd SEQ ID NO: 35 (5′-GGGAGCAGGGGCCAGTACAG-3′) and IL-1 Orev (5′-GGGAAGCTTTTAGCTTT TCATTTTGATCATC).

### Example 12

### Preparation of Plsamid pInt-MSH and Other pInt Vectors

pInt-MSH was constructed by subcloning the expression cassette (from beginning of GPD promoter to end of the CYC1 transcription termination sequence) on a SacI/KpnI fragment onto the same sites in pFL34. All other pint vectors were constructed by PCR amplification of the corresponding expression cassettes and cloning into the HindIII/KpnI sites of pFL34. Oligonucelotides used for PCR amplification were GPDFWD SEQ ID NO 36: (5′-CCCAAGCTTTTACCATCACCGTCACC-3′) and CYC1REV SEQ ID NO 37: (5′-CCCGGTACCGTCATGTAATT AGTTATGTC-3′). Digestion of pInt-MSH and other pint vectors within the URA3 gene will provide sequences on both ends of the linear DNA that are homologous to the chromosomal ura-3 gene (mutant ura-3 strain, see FIG. 7A). The homologies at the ends of the linearized DNA mediate homologous recombination into the ura-3 locus and give rise to a Ura⁺ prototrophic phenotype.

### Example 13

### Integration of a PCR Product

To facilitate chromosomal integration (schematic diagram in FIG. 7B), each expression cassette was PCR amplified (from beginning of GPD promoter to beginning of the URA3 gene) using the following oligonucelotides: UPINT (SEQ ID NO 38)(5′-CGTGCTTCTGGTACATACTTGCAATTTATACAGTGA TGACCGCTGGACCATGATTA CGCCAAG-3′) and DWNINT (SEQ ID NO 39) (5′-TTTAGCATGGCCATTGAATGTAACAATTATATATATCGCMGCACGATTCGGTAATC TCCGAG -3′). UPINT comprises a 45 bp sequence complementary to +1542 through +1587 sequences of the HO gene, followed by the first 18 base pairs of the GPD promoter. DWNINT contains a 45 bp sequence corresponding to -2680 through -2635 sequences of the HO gene (+1 being the ATG codon) followed by 18 base pairs complementary to the beginning of the URA3 gene. The resulting PCR product will have flanking sequences homologous to the HO gene, which will facilitate chromosomal integration of the expression cassette at the HO locus upon a double cross-over event. The HO gene encodes a site-specific endonuclease, which is required for mating-type switching, and its absence has no known effect on the physiology of yeast. The chromosomal integration event will render the yeast cells a Ura⁺ prototrophic phenotype, allowing selection for recombinants in the absence of uracil.

### Example 14

Construction of pSYMB6, a Vector With a ThyA Selection System

To construct the clinical grade vector pSYMB6, initially the ThyA gene was PCR amplified from Lactobacillus casei chromosomal DNA using primers thyANsilFor SEQ ID NO: 40 (5′-CCAATGCATGGCACAGCTTGATGCGATC-3′), and thyANsilRev SEQ ID NO: 41 (5′-CCAATGCATGTG TCATTGGTAAACCTGAC -3′). The resulting PCR product was digested with Nsil and cloned into similarly digested pSYM3 to obtain plasmid pSYMB5. pSYMB5 constructs were selected in an E. coli thyA deletion strain (MM21). To obtain pSYMB6, the Erymthromycin-resistance gene (Em) was deleted from the plasmid by a long-range PCR strategy in which the Em gene was excluded from the final PCR product. This was accomplished by designing the PCR primers to hybridize to the ends of the Em gene and direct the polymerization reaction to point away from the Em gene. Following a Dpnl digestion (to remove the template DNA), the PCR product was circularized in a ligation reaction and transformed into the MM21 ThyA deficient E. coli. Transformants were selected on thymine deficient media.

### Example 15

### Isolation of ThyA Mutant Strains of Lactobacillus and Lactococcus

Strains of Lactobacilli and Lactococci can be constructed in two ways: by selection for thyA chromosomal mutants or by deletion of the thyA gene from the chromosome. ThyA chromosomal mutants can be isolated by plating cells on solid modified MRS or M17 media containing trimethoprim (20-400 μg/ml) and thymidin or thymin (50-100 ug/ml). Although wild-type cells are sensitive to the antibiotic trimethoprim, thyA mutants are resistant and can grow in the above mentioned media.

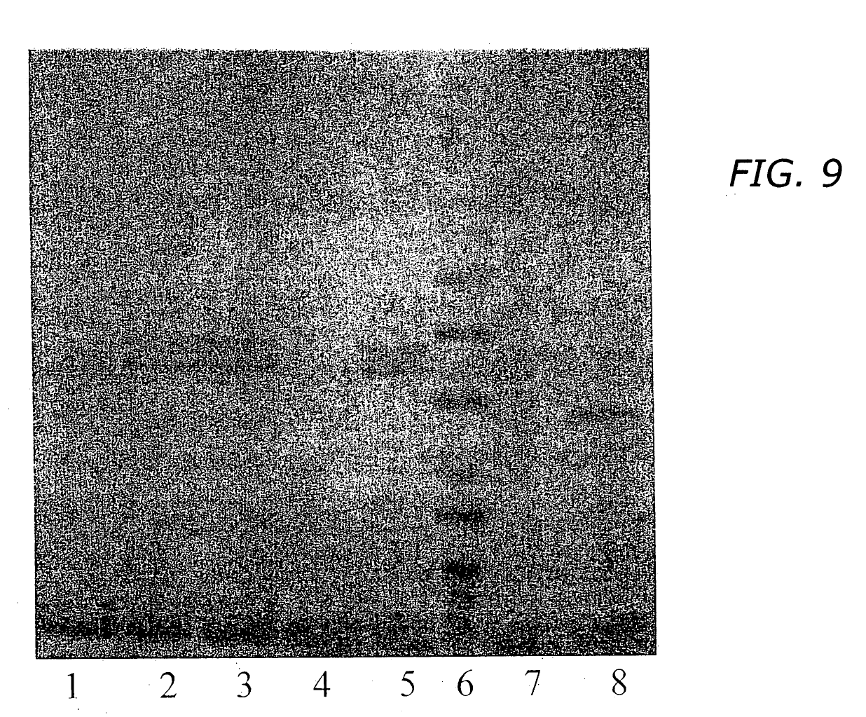

Chromosomal deletion of the ThyA gene is performed by replacing the ThyA ORF with sequences encoding a reporter gene. Examples of reporter genes are GFP, Luciferase and ,-galactosidase. Thus, a fusion construct containing ThyA regulatory sequences (promoter and 3′ untranslated region) flanking a reporter gene will be cloned into an integrative vector such as, but not limited to, pHY304 ( for a description of these constructs see below). This vector has a temperature-sensitive origin of replication and the Em gene as selectable marker; once the vector has been transformed into the cells at the permissive temperature, it can be targeted to the chromosome by incubation of the cells at the non-permissive temperature and selection for erythromycin resistance. Targeting into the chromosome will be directed by homologous recombination between ThyA flanking sequences on the plasmid and the ThyA gene on the chromosome (See FIG. 9). The Em resistant cells will be screened for GFP expression (GFP used as an example of a reporter gene) by fluorescence microscopy, and correct integration of the plasmid will be confirmed by diagnostic PCR amplification of chromosomal DNA. As a result of the chromosomal integration, ThyA flanking sequences are duplicated on the chromosome, which provides an opportunity for an intrachromosomal recombination event leading to the excision of the plasmid sequences, and a 50% chance of replacing the chromosomal thyA gene with the GFP-fusion gene (GFP driven by ThyA regulatory sequences, see FIG. 9). Such intrachromosomal recombinants will be resistant to the lethal effect of Trimethoprim due to the deletion of the thyA gene. Thus, to derive intrachromosomal recombination and to select for thyA deletion strains, Em-resistant GFP positive cells will be incubated in the presence of Trimethoprim and thymidine at the temperature permissive for replication of the integrative vector. Next, Trimethoprim resistant cells will be screened for expression of GFP and sensitivity to erythromycin, which should represent cells that have lost the plasmid. Finally, correct replacement of the chromosomal ThyA gene with the GFP-fusion gene will be determined by diagnostic PCR of chromosomal DNA.

### Example 16

Construction of ThyA Integration Vector for L. lactis (pSYMB7)

The ThyA gene plus 200 bp flanking sequences will be PCR amplified from L. lactis genomic DNA and cloned into pUC19. Next, the ThyA ORF will be deleted from this pUC19 construct by a long range PCR using primers that flank and point away from the ThyA ORF. The upstream and downstream primers will also carry on their 5′-ends sequences complementary to the beginning and the end of GFP ORF, respectively. Next, the resulting PCR product will be transformed along with a second PCR fragment corresponding to the GFP ORF, into a RecA⁺ bacterial host such as, but not limited to, DH5α. Transformant colonies will form upon successful homologous recombination between the two PCR products within the GFP sequences, and generation of a circular plasmid. As a result of this homologous recombination, GFP will be expressed under the control of ThyA regulatory sequences. Finally, to construct the ThyA integration vector, GFP ORF along with 200 bp ThyA flanking sequences will subcloned into the integration vector pHY304.

### Example 17

### Construction of ThyA Integration Vector for LAB (pSYMB8)

ThyA ORF along with 70 bp flanking sequences will be PCR amplified from LAB chromosomal sequences and cloned into pUC19. Next, an internal fragment of ThyA ORF will be removed by restriction digestion, and will be replaced with a GFP PCR fragment with compatible ends. In the resulting construct, the GFP ORF will be in frame with the ThyA ORF at the 5′ end and carry a stop codon at the 3′-end, out of frame with the remaining ThyA ORF sequences downstream. The resulting ThyA-GFP fusion along with ThyA flanking sequences will be subcloned into pHY304 to obtain a LAB ThyA integration construct.

### Example 18

### Integration of LAB and L. lactis Expression Constructs Into the Chromosome

The strategy used for construction of ThyA mutants can be used for integration of LAB and L. lactis expression cassettes into the chromosome. Briefly, expression cassettes will be PCR amplified from the respective expression constructs (such as pSYMB3, pSYMB4 etc.) and cloned in the middle of ThyA flanking sequences. The resulting integration construct can be used to replace the chromosomal ThyA gene as described above.

### Examples 19-23

### Protein Expression in Clinical Vectors

### Example 19

### Expression in Caulobacter Crescentus

Plasmid pCX-MSH or pCX-VP7 was transformed into Caulobacter crescentus by electroporation. Single colony of transformants were inoculated into 5 ml of PYE medium containing 2 μg/ml chloramphenicol, and grown at 30° C. for 16-18 hours. The next day, the overnight culture was diluted 25 fold into M11 expression medium containing 2 μg/ml of chloramphenicol. These diluted cultures were grown at 30° C. for 2 days with gentle shaking (80-100 rpm), and samples were harvested at regular intervals to test for expression of target protein.

### Example 20

### Expression in Escherichia coli

Inoculate the expression E.coli strain into IMC medium containing 100 μg/ml of ampicillin; grow at 25° C. with shaking overnight. Add 1×10¹⁰ cells of overnight culture to 50 ml of IMC medium containing 100 μg/ml ampicillin and 100 μg/ml of tryptophan. Grow the culture at 25° C., taking samples at every hour, check cell density at O.D. 600 nm, centrifuge to collect the cells and test for expression by SDS-PAGE, ELISA, and Western Blot.

### Example 21

### Expression in LAB

Inoculate the expression Lactobacilli strain into modified MRS medium, grow at 37° C. without shaking overnight. Make a 1/50 dilution of the overnight culture into 50 ml of modified MRS medium. Grow the culture at 37° C., take samples at every hour, centrifuge to separate cells and supernatant, and test both for expression by ELISA. In addition, assay both fractions for biological activity. In addition, measure and record the turbidity of the culture at OD600 n.m. to correlate level of expression with growth phase.

### Example 22

### Expression of Proteins on Yeast Cell Surface

EBY 100 yeast transformed with pYD1 or pYD1-based expression vectors were grown overnight at 30° C. in YNB-CAA medium containing 2% glucose. Cells were harvested by centrifugation and resuspended in YNB-CAA medium containing 2% galactose to an OD₆₀₀ of 0.5˜1. Cells were grown at 20˜25° C., and samples were harvested at regular time intervals to analyze for expression by immunofluorescent staining.

### Example 23

### Protein Expression in Yeast:

Yeast expressing a cell wall displayed GFP protein was grown to mid log phase, and aliquots at various cell densities were harvested (cell density measure by absorbance at 600 nm). Yeast transformed with empty vector (PGPDDSPLY, see below) was also harvested as control. An equivalent of 2×10⁷ cells was pelleted, washed and boiled in SDS-polyacrylamide gel (PAGE) loading buffer. Proteins were separated on a 4-12% Novex gradient gel, transferred to a nitrocellulose membrane, and blotted with a monoclonal GFP antibody (mAb11E5, Qbiogene). Antigenic proteins were visualized by treating the membrane with a secondary Horse radish peroxidase(Hrp)-conjugated anti-mouse antibody, followed by addition of a chromogenic Hrp substrate. As shown in FIG. 10, only yeast transformed with GFP expression constructs showed protein bands recognized by the anti-GFP antibody. The presence of multiple anti-GFP reactive bands, in addition to the large full-length product, suggest proteolytic degradation of the gene product.

### Examples 24-27

### Characterization of the Expression Product

### Example 24

### Characterization Using SDS-PAGE

Collect cells (for surface expression) or the protein aggregate in the medium (for pCX) by centrifugation, resuspend in sample loading buffer for SDS-PAGE and heat at 95° C. for 10 min. Fractionate the samples on SDS-Polyacrylamide gel electrophoresis (SDS-PAGE). Stain the gel with Commassie brilliant blue to detect proteins.

### Example 25

### Characterization Using Western Blot

Protein samples fractionated by SDS-PAGE were transferred onto nitrocellulose membranes by electroblotting. Protein-containing membranes were treated with antigen-specific primary antibodies. The presence of the antigen-antibody complexes were identified by exposing to a secondary antibody that recognizes the antigen-specific antibody and is linked to enzyme. Next, incubation of the membrane with substrates for the antibody-linked enzyme will generate either color or light energy, which allow the detection of the protein of interest.

### Example 26

### Characterization Using ELSIA

Collect cells (for surface expression) or the protein aggregate in the medium (for secreted expression ) by centrifugation, resuspend in coating buffer, and coat onto wells of an ELISA plate. Treat wells with primary antigen-specific antibody, wash, and treat with secondary enzyme linked antibody. To detect protein expression, add substrate for the linked enzyme and monitor color development on an ELISA plate reader.

### Example 27

### Detection of Surface-Expressed Proteins

Harvest cells transformed with surface-expression constructs or empty expression vector, wash with PBS; incubate with corresponding antibody at 4° C. for 30 min. Centrifuge and wash with PBS; incubate at 4° C. for 30 min in the dark with secondary antibody conjugated with fluorescein isothiocyanate (FITO). Wash the cells two times with PBS, and observe the result under fluorescent microscope.

Present gene therapy techniques rely on vectors that are either immunologically active such as viruses, naked plasmid DNA, or artificial vectors such as liposomes. Each of these gene vectors has advantages and disadvantages. Viral vectors can provoke immune responses that are either immediately injurious to the recipient or elicit an immune response that limits, or prevents, further administration of the same vector. Naked plasma DNA has a very low cellular uptake efficacy and are vulnerable to the recipient's nucleases. Liposomes have limited applications due to the complexity associated with manufacturing, low transfection efficiency and low rates of stable integration. Table 4 below summarizes the relative advantages and disadvantages of present gene therapy vector systems including the microflora vectors of the present invention.

Several of the most significant advantages of the vectors of the present invention include their low immunogencity to the recipient, the large plasmid inserts that can be transported, ease of scale-up in FDA approved manufacturing environments and the associated safety to manufacturing personnel as well as healthcare professionals due to their non-pathogenic character and the ease with which specific tissues and cell types can be targeted.

In one embodiment of the present invention the clinical grade vectors are used to deliver a therapeutic protein directly to a site in need of treatment. The entire vector system and recombinant therapeutic protein can be delivered simultaneously because there are no health risks (i.e. infection) associated with the vector. This simplifies the process by eliminating the need for costly post production purification to remove the recombinant protein expression system and provides for in situ production of the therapeutic protein simultaneously.

Furthermore, when used to deliver immunogenic compositions to an animal oral administration permits immune cell targeting by exposing the M cells of the intestines directly to the antigen expressing/secreting vector. In addition, collateral heath benefits may be experienced in recipients of the orally delivered vectors due to the probiotic effects associated with many of the organisms that can be used in accordance with the teachings of the present invention (Fuller, R. 1993. Probiotic foods—current use and future developments. IFI NR 3: 23-26; Mitsuoka, T. 1984. Taxonomy and ecology of Bifidobacteria. Bifidobacteria Microflora 3: 11; Gibson, G. R. et al., 1997. Probiotics and intestinal infections, p.10-39. In R. Fuller (ed.), Probiotics 2: Applications and practical aspects. Chapman and Hall, London, U.K.).

TABLE 4
Gene Therapy Vector Systems Compared

| Vector System | Advantaged | Disadvantages |
|---|---|---|
| Retroviruses | Long lasting gene | Only infects dividing |
| | expression | cells |
| Lentiviruses | Long lasting gene | Reputation for |
| | expression | being quite deadly |
| | Will infect non-dividing cells | |
| Adenoviruses | Will infect non-dividing cells | Very immunogenic - |
| | High rate of delivery | leading to transient |
| | | gene expression |
| Adeno-associated | Much less immunogenic | Difficult to produce |
| viruses | than adenovirus | in high quantities |
| | Long term expression | |
| | possible | |
| Herpes virus | Can carry a great deal of | Immunogenic and |
| | DNA | potentially toxic |
| Liposomes | Not immunogenic | Low rate of delivery |
| | Can deliver large quantities | Transient |
| | of DNA | expression |
| Naked DNA | No viral component | Transient gene |
| | | expression |
| | | More difficult to |
| | | target to specific |
| | | tissues |
| Microflora Vector | Non-immunogenic, | None presently |
| of the Present | Can carry large DNA | identified |
| invention | payloads, | |
| | Easy to propagate in large | |
| | quantities, | |
| | Non-pathogenic | |
| | Collateral health benefits | |
| | (probiotic) | |
| | Cell targeting for vaccines | |
| | (M Cells) | |

One embodiment of the present invention is a method of using clinical grade vectors described herein to treat or palliate traumatic ocular inflammation and uveitis. Uveitis is inflammation of the uvea, the middle layer of the eye between the sclera (white outer coat of the eye) and the retina (the back of the eye). The uvea contains many of the blood vessels that nourish the eye. Inflammation of this area, therefore, can affect the cornea, the retina, the sciera, and other important parts of the eye. Uveitis occurs in acute and chronic forms, and affects men and women equally. It can happen at any age, but occurs primarily between the ages of 20 and 50, and most commonly in one's 20s. Although the exact cause of uveitis is often unknown, it may result from trauma to the eye, as in the case of chemical exposure. In addition, uveitis may be caused by a viral infection (for example, cytomegalovirus, as seen in patients with AIDS), a fungal infection (such as histoplasmosis), or an infection caused by a parasite (such as toxoplasmosis; a newborn may develop uveitis if the mother was exposed to toxoplasmosis during pregnancy). Uveitis is also associated with underlying immune-related disorders, including Reiter's syndrome, multiple sclerosis, juvenile rheumatoid arthritis, Crohn's disease, and sarcoidosis. Certain diseases—including leukemia, lymphoma, and malignant melanoma—may have symptoms that resemble uveitis. Some medications, such as rifabutin, cidofovir, pamidronic acid, and sulfonamides, may cause uveitis. In many cases, an underlying cause is not identified. (Alexander K L, et al. 1997 . Optometric Clinical Practice Guideline: Care of the Patient with Anterior Uveitis. 2nd ed. American Optometric Association.)

Current treatment options include corticosteroids to reduce swelling and pain, cycloplegics (such as cyclopentolate and homatropine) to reduce pain, antimicrobials to treat infection, anti-inflammatories to reduce swelling and medications to suppress the immune system (Berkow R, Fletcher A J, Beers M H, eds. The Merck Manual. Rahway, N.J.: Merck & Co.; 1992: 2380-2382).

The present invention demonstrates that uveitis in animals can be inhibited and/or treated using αMSH expressing vectors made in accordance with the teachings of the present invention. Traumatic uveitis and endotoxin induced uveitis (EUI) may be effectively treated using the clinical grade microflora vectors of the present invention including, but not limited to, Lactic Acid Bacteria and yeast which express recombinant αMSH (rαMSH).

### Example 28

### Treating Surgically Induced Uveitis Using a Clinical Grade Vector Expressing rα MSH

A. Experimental Design:

Surgical non-perforating incisions were made to the cornea of rats. Treatment of recombinant Microorganisms which express αMSH was given to the rats either topically or orally. Evidence of uveitis severity was determined by assessing various parameters including hyperemia, edema, aqueous protein levels and the number of inflammatory cells in the aqueous humor, as well as their number determined histologically in the injured cornea. Each parameter was assessed at 24 hours post injury. The study used 8 groups of rats. Group 1: normal control, normal, untreated rats (10 rats); treatment group 2: treatment control: surgically induced uveits rats treated daily by topical application of 0.45% saline tid (10 rats); group 3: Yeast oral treatment group: surgically induced uveits rats treated orally with yeast expressing rαMSH (10¹⁰ yeast/ml qd) (5 rats); group 4: Yeast topical treatment group: surgically induced uveits rats treated topically with yeast expressing rαMSH (10¹⁰ yeast/ml qd) (5 rats); group 5: LAB oral treatment group: surgically induced uveits rats treated orally with LAB expressing rαMSH (10¹⁰ bacteria/ml qd) (5 rats), group 6: LAB topical treatment group: surgically induced uveits rats treated topically with LAB expressing rαMSH (10¹⁰ bacteria/ml qd) (5 rats); LAB control group: normal rats treated topically with LAB expressing recombinant (αMSH (rαMSH) (10¹⁰ bacteria/ml qd) (4 rats); yeast control group: normal rats treated topically with yeast expressing rαMSH(10¹⁰ yeast/ml qd) (4 rats).

B. Materials and Methods:

1. Animals:

Lewis rats of either sex, weighing 125 g to 250 g.

2. Clinical grade Vectors

a. Yeast

Saccharomyces cerevisiae yeast strain W303-1A transformed with p426GPD or pLongα-sp-MSH.

b. LAB

Lactobacillus casei transformed with pSYMB4 or pSYMB.

3. Vector Preparation

Transformed and non-transformed yeast and LAB were grown on rich solid media, and harvested during log phase (1 day before cells reach maximum colony size), washed in PBS and resuspended in PBS at a concentration of 10¹⁰ cells/ml. Aliquots of this final suspension were used for administration to animals.

4. Study Outcome Parameters used to measure study outcome:

a. Protein concentration and inflammatory cell count:

Protein was measured using the Lowry technique, while inflammatory cells were counted using a Coulter cell counter. The difference between animals treated with (αMSH and control determines the effectiveness of αMSH in controlling post traumatic inflammation.

b. Histopathology

At 24 hours post trauma, following aqueous humor withdrawal, the eye are enucleated. Areas of interests include cornea, sclera, and the iris ciliary body. The tissue was fixated with glutaraldehyde 2%.

After a drying process the cornea was cut and stained with H&E and PAS. The difference between animals treated with αMSH and control demonstrates the effect of αMSH.

c. Clinical assessment:

Conjunctival hyperemia, edema, hemorrhages, and discharge, as well as corneal changes 24 hours following ocular trauma were assessed using the operating microscope. A grade scale of 1 to 4 will be used; 1 being mild and 4 being severe. Additional evaluation of hyperemia was done morphometrically using a digital camera.

### Example 29

### Treating Endotoxin Induced Uveitis Using a Clinical Grade Vector Expressing rαMSH

A. Experimental Design:

Uveitis will be induced in rats by injection of Salmonella typhimurium LPS endotoxin into the hind footpad of the animals. Treatment of αMSH will be given to the rats either topically or intramuscularly. Evidence of uveitis severity will be determined by assessing various parameters including hyperemia, edema, aqueous protein levels and the number of inflammatory cells in the aqueous humor, as well as their number determined histologically in the injured cornea. Each parameter will be assessed at 1 h, 3 h, 6 h, 12 h, and 24 h after treatment of αMSH . The study will use at least 8 groups of rats. Group 1: normal control, normal, untreated rats (10 rats); treatment group 2: treatment control: surgically induced uveits rats treated daily by topical application of 0.45% saline tid (10 rats); group 3: Yeast oral treatment group: surgically induced uveits rats treated orally with yeast expressing rαMSH (10¹⁰ yeast/ml qd) (5 rats); group 4: Yeast topical treatment group: surgically induced uveits rats treated topically with yeast expressing rαMSH (10¹⁰ yeast/ml qd) (5 rats); group 5: LAB oral treatment group: surgically induced uveits rats treated orally with LAB expressing rαMSH (10¹⁰ bacteria/ml qd) (5 rats), group 6: LAB topical treatment group: surgically induced uveits rats treated topically with LAB expressing rαMSH (10¹⁰ bacteria/ml qd) (5 rats); LAB control group: normal rats treated topically with LAB expressing recombinant αMSH (rαMSH) (10¹⁰ bacteria/ml qd) (4 rats); yeast control group: normal rats treated topically with yeast expressing rαMSH(10¹⁰ yeast/ml qd) (4 rats). Because this is an acute inflammation model, pretreatment with αMSH maybe needed if tissue damage occur too earlier and too severe.

B. Materials and Methods:

1. Animals:

Lewis rats of either sex, weighing 125 g to 250 g.

2. Clinical grade Vectors

a. Yeast

Saccharomyces cerevisiae yeast strain W303-1A transformed with P426GPD or pLongα-sp-MSH.

b. LAB

Lactobacillus casei transformed with pSYMB4 or PSYMB.

3. Vector Preparation

Transformed and non-transformed yeast and LAB are grown on rich solid media, and harvested during log phase (1 day before cells reach maximum colony size), washed in PBS and resuspended in PBS at a concentration of 10¹⁰ cells/ml. Aliquots of this final suspension will used for administration to animals.

4 Study Outcome Parameters used to measure study outcome:

a. Protein concentration and inflammatory cell count:

Protein will be measured using the Lowry technique, while inflammatory cells were counted using a Coulter cell counter. The difference between animals treated with αMSH and control determines the effectiveness of αMSH in controlling post traumatic inflammation.

b. Histopathology

At 24 hours post trauma, following aqueous humor withdrawal, the eye are enucleated. Areas of interests include cornea, sclera, and the iris ciliary body. The tissue was fixated with glutaraldehyde 2%.

After a drying process the cornea was cut and stained with H&E and PAS. The difference between animals treated with αMSH and control demonstrates the effect of αMSH.

c. Clinical assessment:

Conjunctival hyperemia, edema, hemorrhages, and discharge, as well as corneal changes 24 hours following ocular trauma are assessed using the operating microscope. A grade scale of 1 to 4 will be used; 1 being mild and 4 being severe. Additional evaluation of hyperemia was done morphometrically using a digital camera.

d. Cytokines and Polymorphonuclear leukocytes (PMNs) of Aqueous humor

Levels of TNF-α are determined by a cytotoxicity assay, levels of IL-1, IL-2, IL-6 and IFN-γ are determined by radioactive isotope.

The numbers of PMNs are counted under the microscope. The activity of PMNs is determined using a modification of the method described by Williams R N. (curr Eye Res 2: 465, 1983) Levels of αMSH aqueous humor are assayed by ELISA.

### Example 30-3

### Cell Targeting Using Clinical Grade Vectors

M cells are specialized epithelial cells in the gut that mediate transport of macromolecules, viruses and the like from the lumen of the gut to underlying lymphoid tissue called peyer's patches. In addition to serving as a first line of defense against foreign organisms and macromolecules, M cells vesicular transport provides a gateway for therapeutic compounds to the blood stream. Thus the bacterial and yeast vehicles will be engineered to carry M-cell targeting molecules on their surface.

In one embodiment the LAB contains a construct coding for an M cell targeting factor. This factor may be included in the plasmid containing the heterologous gene or it may be on a separate plasmid. Upon expression, the M cell targeting factor allows the LAB to preferentially bind to M cells over other forms of epithelial cells. There are in general three types of elements which have been studied to improve drug binding capabilities to target M-cells (Chen et al. U.S. Pat. No. 6,060,082) (Ginkel et al. CDC. 6(2), 2000). One is lectin, which can be incorporated into a cell's surface (see for example U.S. Pat. No. 6,060,082). The second is the sigma protein from reovirus, which targets M cell factors and may be expressed as a fusion protein (Wu, Y., et al., “M cell-targeted DNA vaccination” Proc. Natl Acad. Sci. USA 98(16): 9318-23 (2001)). The third method involves the development and use of monoclonal antibody fragments targeted specifically, or at least predominantly to M-cells. In one embodiment of the present invention the reovirus sigma protein is expressed on the LAB cell surface along with the therapeutic protein.

Further M-cell targeting embodiments of the present invention include screening for LAB that preferentially bind to epithelial cell in-vitro and use these strains to produce the clinical grade vectors of the present invention. In other embodiments of the present invention the Lactobacillus and/or Saccharomyces organisms are provided with adhesins proteins from bacteria and viruses that target M cells, such as the Yersinia species and Salmonella typhi, respectively. (Clark, M. A., et al., “M-cell surface P 1 integrin expression and invasin-mediated targeting of Yersinia pseudotuberculosis to mouse Peyer's patch M cells” Infect Immun. 66: 1237-43 (1998); Baumler, A. et al., “The Ipf fimbrial operon mediates adhesion of Samonella typhirium to murine Peyer's patches” Proc. Natl. Acad. Sci. USA 93: 279-83 (1996). Such bacterial and viral adhesins are proteins that mediate M cell binding.

The M cell targeting compounds described above can be incorporated into the cell wall of the modified Lactobacillus. This can be accomplished by adding the M cell targeting compound to modified Lactobacillus protoplasts that are regenerating cell walls. In a preferred embodiment, the M cell targeting compound will be derivatized to lipids designed to act as membrane anchors . Such functionalized lipids can be purchased from Avanti Polar Lipids, Inc. (Alabaster, Ala.). Alternatively, a plasmid in the modified Lactobacillus organism could encode an M cell targeting polypeptide. In one embodiment the plasmid containing the sequence for the antigen would also contain the sequence for the M cell targeting polypeptide. In this embodiment, the M cell targeting polypeptide could be attached to the sequence for the antigen. Alternatively, the M cell targeting polypeptide sequence could be attached to surface binding promoter regions and operably linked to a promoter region, such that expression of the plasmid would produce two heterologous proteins. In an alternate embodiment, a second plasmid would contain the M cell targeting polypeptide sequence attached to surface binding promoting regions and operably linked to a promoter, such that the vector would harbor two different recombinant plasmids.

In an additional embodiment, the plasmid containing the heterologous genetic element may also contain the polynucleotide sequence coding for a synthetic peptide containing an oc integrin-binding motif (arginine-glycine-aspartic acid, RGD) fused to the sequence coding for the heterologous genetic element, for the enhancement delivery. It has been shown that integrin proteins are capable of binding the RGD motif are located on the apical surface of a polarized human bronchial epithelial cells. Scott, E. S., et al., “Enhanced Gene Delivery To Human Airway Epithelial Cells Using An Integrin-Targeting Lipoplex” The Journal Of Gene Medicine 3: 125-134 (2001). Receptor-ligand interaction is between peptides containing the RGD (arginine-glycine-aspartic acid) motif and several members of the integrin family of cell surface receptors have been well-characterized. Hence, in this approach receptor-mediated endocytosis is used to gain entry to the target epithelial cells. Scott, E. S., et al., “Enhanced Gene Delivery To Human Airway Epithelial Cells Using An Integrin-Targeting Lipoplex” The Journal Of Gene Medicine 3: 125-134 (2001) and also, Hart, S., et al., “Gene Delivery And Expression Mediated By An Integrin-Binding Peptide” Gene Ther. 2: 552-554 (1995)

An additional strategy for directing bacteria and yeast vehicles to the mucosal surfaces is to target them for binding to the monosialoganglioside GM1, which is present on the epithelial cells of mucosal surfaces. GM1 is normally concentrated in regions of the plasma membrane called rafts, which are sphingolipid and cholesterol-rich patches that function as membrane trafficking and surface signaling regions (Simons K., and Ikonen E., 1997, “Functional rafts in cell membranes”, Nature 387: 569-572). Indeed, GM1 is the primary target of the Cholera Toxin (Ctx) of Vibrio Cholera, and E. coli enterotoxin (Etx) (Lencer W I, Hirst T R, and Holmes R K, 1999, “Membrane traffic and cellular uptake of cholera toxin”, Biochim Biophys Acta 1450: 177-190). Ctx and Etx are composed of five identical B subunits and a single A subunit, with the B subunit oligomer (CtxB and EtxB) functioning as the receptor for GM1. CtxB or EtxB binding induces GM1 cross-linking, which leads to endocytosis of toxin-GM1 complexes and eventual delivery of the A subunit enzyme to the cytosol (Lencer W I, Hirst T R, and Holmes R K, 1999, “Membrane traffic and cellular uptake of cholera toxin”, Biochim Biophys Acta 1450: 177-190).

In the absence of the A subunit, CtxB is non-toxic and it can form an independent pentameric complex which is capable of binding GM1. Thus, purified CtxB has been used as a tool for delivery of CtxB-coupled antigens to mucosal surfaces (George-Chandy et al. 2001, “Cholera toxin B subunit as a carrier molecule promotes antigen presentation and increases CD40 and CD86 expression on antigen-presenting cells” Infect. Immun. 69: 5716-25; Sadeghi et al. 2002, “Genetic fusion of human insulin B-chain to the B-subunit of cholera toxin enhances in vitro antigen presentation and induction of bystander suppression in vivo” Immunology 106: 237-45). CtxB has also been expressed on the surface of non-pathogenic E. coli and Staphylococci as a means of developing live bacterial vaccine delivery systems for administration by the mucosal route (Liljeqvist et al. 1997, “Surface display of the cholera toxin B subunit on Staphylococcus xylosus and Staphylococcus carnosus”, Appl. Env. Microbiol. 63: 2481-2488; Klauser et al. 1990, “Extracellular transport of cholera toxin B subunit using Neisseria IgA protease β-domain: conformation-dependent outer membrane translocation” EMBO J. 9: 1991-1999; Klauser et al. 1992, “Selective extracellular release of cholera toxin B subunit by Esherichia coli:dissection of Neisseria Iga_{β}-mediated outer membrane transport” EMBO J. 11: 2327-2335). In the case of Staphylococci surface expressions, EtxB targeted to the outer membrane was shown to form a functional complex capable of binding GM1 in vitro (Liljeqvist et al. 1997, “Surface display of the cholera toxin B subunit on Staphylococcus xylosus and Staphylococcus carnosus”, Appl. Env. Microbiol. 63: 2481-2488). Thus, in order to take advantage of this highly specific and efficient mucosal delivery system, our yeast and bacterial delivery vectors will be engineered to surface display EtxB.

An alternative method for targeting delivery vectors is to express on their surface, proteins that have been shown to mediate binding to epithelial cells. Such proteins have been identified in the pathogenic yeast Candida albicans (Fu et al., Expression of the Candida albicans gene ALS1 in Saccharomyces cerevisiae induces adherence to endothelial and epithelial cells. Infection and Immunity, 66: 1783-1786) and Candida glabrata (An adhesin of the yeast pathogen Candida glabrata mediating adherence to human epithelial cells. Science, 285: 578-582). Expression of these epithelial-targeting proteins on the surface of Saccharomyces cerevisiae confers epithelial-cell binding to this naturally non-adherent organism.

In addition to these methods, vector cell-wall anchoring domains or yeast cell-wall proteins can be used which include the α-agglutinin gene (AGα-1), Cell wall protein 2 (CWp2p), Sed1p and others as outlined by Van Der Vaart et al. (Comparison of cell wall proteins of Saccharomcyes cerevisiae as anchors for cell surface expression of heterologous proteins, Appl. Env. Microbiol. 63: 615-620, 1997).

### Pharmaceutical Compositions Incorporation the Microflora Vectors of the Present Invention

The clinical grade vectors of the present invention can be administered over a wide range of concentrations depending on the route of administration selected (oral or topically). However, generally the pharmaceutical compositions of the present invention contain from approximately 10³ to approximately 10¹¹ viable microflora vectors per unit dose in a pharmaceutically acceptable carrier. Solid formulations of the compositions for oral administration may contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, without limitation, microcrystalline cellulose, corn starch, sodium starch glycolate, and alginic acid. Tablet binders that may be used include acacia, methylcellulose, sodium carboxymethylcellulose, polyvinylpyrrolidone (Povidone™), hydroxypropyl methylcellulose, sucrose, starch, and ethylcellulose. Lubricants that may be used include magnesium stearates, stearic acid, silicone fluid, talc, waxes, oils, and colloidal silica.

Liquid formulations of the compositions for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

A topical liquid and semi-solid ointment formulation typically contains a concentration from approximately 10³ to approximately 10¹¹ viable microflora vectors in a carrier such as a pharmaceutical cream base. Various formulations for topical use include drops, tinctures, lotions, creams, solutions, and ointments containing the active ingredient and various supports and vehicles. The optimal percentage of the clinical grade vectors of the present invention in each pharmaceutical formulation varies according to the formulation itself and the therapeutic effect desired in the specific pathologies and correlated therapeutic regimens.

Conventional methods, known to those of ordinary skill in the art of medicine, can be used to administer the pharmaceutical formulation(s) to the patient. Typically, the pharmaceutical formulation will be administered to the patient orally in a liquid, tablet or capsule form. For topical applications the pharmaceutical compositions will be applied as a liquid, cream or using a transdermal patch containing the pharmaceutical formulation. Transdermal patches are left in contact with the patient's skin (generally for 1 to 5 hours per patch). Other transdermal routes of administration (e.g., through use of a topically applied cream, ointment, or the like) can be used by applying conventional techniques. The pharmaceutical formulation(s) can also be administered via other conventional routes (e.g. oral, subcutaneous, intrapulmonary, transmucosal, intraperitoneal, intrauterine, sublingual, intrathecal, or intramuscular routes) by using standard methods. In addition, the pharmaceutical formulations can be administered to the patient via injectable depot routes of administration such as by using 1-, 3-, or 6-month depot injectable or biodegradable materials and methods. 4In one embodiment of the present invention an animal is provided with a single dose containing from approximately 10³ to 10¹¹ viable microflora organisms per gram of therapeutic or prophylactic composition. The total amount consumed will depend on the individual needs of the animal and the weight and size of the animal. The preferred dosage for any given application can be easily determined by titration. Titration is accomplished by preparing a series of standard weight doses each containing from approximately 10³ to 10¹¹ vectors per unit dose. A series of doses are administered beginning at 10³ vectors and continuing up to a logical endpoint determined by the size of the animal and the dose form. The appropriate dose is reached when the minimal amount of vector composition required to achieve the desired results is administered. The appropriate dose is also known to those skilled in the art as an “effective amount” of the clinical grade vector compositions of the present invention.

The effectiveness of the method of treatment can be assessed by monitoring the patient for known signs or symptoms of a disorder. For example, amelioration of ornithine transcarbamylase deficiency and carbamoyl phosphate synthetase I deficiency can be detected by monitoring plasma levels of ammonium or orotic acid. Similarly, plasma citrulline levels provide an indication of argnosuccinate synthetase deficiency, and argnosuccinate lyase deficiency can be followed by monitoring plasma levels of argnosuccinate. Parameters for assessing treatment methods are known to persons of ordinary skill in the art of medicine (see, e.g., Maestri et al., 1991, J. Pediatrics, 119: 923-928). In the case of inflamatory diseases treated with rαMSH such as uveitis, treatment duration and dose can be established by the treating physician by monitoring disease regression using the parameters discussed above. Generally, a therapeutically effective amount of rαMSH is between approximately 1 μg/kg to 100 μg/kg, preferably between approximately 5 μg/kg and 50 μg/kg, even more preferably between approximately 10 μg/kg and 25 μg/kg (μg/kg=μg of active ingredient per kg of host body weight).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A therapeutic compound delivery vector comprising:
an isolated transformed microflora vector having at least one housekeeping gene deleted, or a mutation therein such that said housekeeping gene is inoperable; and
at least one transforming nucleic acid sequence containing at least one gene of interest and an nucleic acid sequence encoding for an operable form of said housekeeping gene.

2. The therapeutic compound delivery vector according to claim 1 wherein said housekeeping gene is thymidylate synthase (thyA).

3. The therapeutic compound delivery vector according to claim 1 wherein said transforming nucleic acid is an extrachromosomal plasmid.

4. The therapeutic compound delivery vector according to claim 1 wherein said transforming nucleic acid is an integrated expression cassette.

5. The therapeutic compound delivery vector according to claim 1 wherein said gene of interest encodes for a protein or polypeptide selected from the group consisting of cytokines and hormones.

6. The therapeutic compound delivery vector according to claim 5 wherein said cytokine is selected from the group consisting of interferons, interleukin (IL)-2 interleukin-4, interleukin-10, interleukin-12, G-CSF, GM-CSF, and EPO.

7. The therapeutic compound delivery vector according to claim 5 wherein said hormone is selected from the group consisting of alpha-melanocyte-stimulating hormone (α-MSH), insulin, growth hormone, and parathyroid hormone.

8. The therapeutic compound delivery vector according to claim 1 where in said vector is a bacteria or yeast.

9. The therapeutic compound delivery vector according to claim 8 wherein said bacteria is selected from the group consisting of Lactobacillus brevis, Lactobacillus casei, Lactobacillus plantarum, Lactobacillus delbrueckii, Lactobacillus delbrueckii subsp. bulgaricus, Lactobacillus helveticus, Lactobacillus pentosus, Lactobacillus fermentum, Lactobacillus amylovorus, Lactococcus lactic, Lactococcus cremoris, Streptococcus sp., Streptococcus gordonii, Escherichia coli and Caulobacter crescentus.

10. The therapeutic compound delivery vector according to claim 8 wherein said yeast is Saccharomyces cerevisiae.

11. A method for treating or palliating an inflammatory disease in an animal comprising:
preparing a transformed microflora vector having at least one housekeeping gene deleted, or a mutation therein such that said housekeeping gene is inoperable and at least one transforming nucleic acid sequence containing at least one gene of interest encoding for an anti-inflammatory compound and an nucleic acid sequence encoding for an operable form of said housekeeping gene and wherein transforming nucleic acid sequence is expressed by said vector;
administering said transformed microflora vector to an animal in need thereof.

12. The method for treating or palliating an inflammatory disease according to claim 11 wherein said anti-inflammatory compound is α-MSH.

13. The method for treating or palliating an inflammatory disease according to claim 11 wherein said transformed microflora vector is a lactic acid bacterium or a yeast.

14. The method for treating or palliating an inflammatory disease according to claim 11 wherein said inflammatory disease is uveitis.

15. The method for treating or palliating an inflammatory disease according to claim 11 wherein said transformed microflora vector is applied topically.

16. The method for treating or palliating an inflammatory disease according to claim 11 wherein said anti-inflammatory compound is secreted.

17. The method for treating or palliating an inflammatory disease according to claim 11 wherein said anti-inflammatory compound is expressed on the surface said transformed microflora vector.

18. A therapeutic compound delivery vector comprising:
an isolated transformed microflora vector having at least one reported gene selected from the group consisting of green fluorescent Protein (GFP), β-alactosidase, amylase, and chloramphenicol acetyl transferase (CAT); and
at least one transforming nucleic acid sequence containing at least one gene of interest.

19. The therapeutic compound delivery vector according to claim 18 where in said vector is a bacteria or yeast.

20. The therapeutic compound delivery vector according to claim 19 wherein said bacteria is selected from the group consisting of Lactobacillus brevis, Lactobacillus casei, Lactobacillus plantarum, Lactobacillus delbrueckii, Lactobacillus delbrueckii subsp. bulgaricus, Lactobacillus helveticus, Lactobacillus pentosus, Lactobacillus fermentum, Lactobacillus amylovorus, Lactococcus lactic, Lactococcus cremoris, Streptococcus sp., Streptococcus gordonii, Escherichia coli and Caulobacter crescentus.


SEQUENCE LISTING (42 sequences)

SEQ ID NO: 1 - DNA, 75 bp, artificial sequence
  oligonucleotide
agatctggtggcggtggctcttattctatggaacattttcgttggggtaaacctgttggtggcggtgcggccgcg

SEQ ID NO: 2 - DNA, 195 bp, artificial sequence
  oligonucleotide
agatctctagatggtggcggtggctcttattctatggaacattttcgttggggtaaacctgttggtggcggtgcggccgcgtcttattctatggaacattttcgttggggtaaacctgttggtggtggcggtggctcttattctatggaacattttcgttggggtaaacctgttggtgagctcgagtaaggatcc

SEQ ID NO: 3 - DNA, 252 bp, artificial sequence
  oligonucleotide
gaattctgaaaaagtctgtcaattttgtttcggcgaattgataatgtgttatactcacaatgaaatgcagtttgcatgcacataagaaaggatgatatcaccgtgaaaaaaaagaaaagtttctggcttgtttcttttttagttatagtagctagtgttttctttatatcttttggatttagcaatcattctaaacaagttgctcaagcggctagcgatacgacatcaactgatcactcaagcaatggtacc

SEQ ID NO: 4 - DNA, 26 bp, artificial sequence
  oligonucleotide
ggggtaccagatctctagatggtggc

SEQ ID NO: 5 - DNA, 30 bp, artificial sequence
  oligonucleotide
cccaagcttggatccttactcgagctcacc

SEQ ID NO: 6 - DNA, 28 bp, artificial sequence
  oligonucleotide
aactgcagtgcaggcacagcttgatgcg

SEQ ID NO: 7 - DNA, 31 bp, artificial sequence
  oligonucleotide
cccaagcttccttttgtgtcattggtaaacc

SEQ ID NO: 8 - DNA, 67 bp, artificial sequence
  oligonucleotide
tgataattattatttaggtgagctttgttgataaaaaggtcttttcaacgtttatgttggggagacc

SEQ ID NO: 9 - DNA, 69 bp, artificial sequence
  oligonucleotide
gtttttcctaacaaaggcctaattttttcaatataaaaaggtctccccaacataaacgttgaaaagacc

SEQ ID NO: 10 - DNA, 28 bp, artificial sequence
  oligonucleotide
cgggatcctgataattattatttaggtg

SEQ ID NO: 11 - DNA, 27 bp, artificial sequence
  oligonucleotide
aactgcaggtttttcctaacaaaggcc

SEQ ID NO: 12 - DNA, 252 bp, artificial sequence; identical to SEQ ID NO: 3
  oligonucleotide

SEQ ID NO: 13 - DNA, 30 bp, artificial sequence
  oligonucleotide
tccccgcggtgaaaaagtctgtcaattttg

SEQ ID NO: 14 - DNA, 28 bp, artificial sequence
  oligonucleotide
gctctagaattgcttgagtgatcagttg

SEQ ID NO: 15 - DNA, 51 bp, artificial sequence
  oligonucleotide
ctagatcttattctatggaacattttcgttggggtaaacctgtttaatgag

SEQ ID NO: 16 - DNA, 51 bp, artificial sequence
  oligonucleotide
gatcctcattaaacaggtttaccccaacgaaaatgttccagagaataagat

SEQ ID NO: 17 - DNA, 29 bp, artificial sequence
  oligonucleotide
tcatctagaaaagcaggggccagtacagc

SEQ ID NO: 18 - DNA, 28 bp, artificial sequence
  oligonucleotide
cccggatccttagcttttcattttgatc

SEQ ID NO: 19 - DNA, 78 bp, artificial sequence
  oligonucleotide
atgagatttccttcaatttttactgcagttttattcgcagcatcctccgcattagctgctggtgcttcttactctatg

SEQ ID NO: 20 - DNA, 60 bp, artificial sequence
  oligonucleotide
ttaaactggcttaccccatctgaagtgttccatagagtaagaagcaccagcagctaatgc

SEQ ID NO: 21 - DNA, 31 bp, artificial sequence
  oligonucleotide
gggaattcatgagatttccttcaatttttac

SEQ ID NO: 22 - DNA, 25 bp, artificial sequence
  oligonucleotide
ggaagcttttaaactggcttacccc

SEQ ID NO: 23 - DNA, 26 bp, artificial sequence
  oligonucleotide
atgagatttccttcaatttttactgc

SEQ ID NO: 24 - DNA, 37 bp, artificial sequence
  oligonucleotide
atagagtaagaagcacctcttttatccaaagataccc

SEQ ID NO: 25 - DNA, 37 bp, artificial sequence
  oligonucleotide
tgttccatagagtaagatcttttatccaaagataccc

SEQ ID NO: 26 - DNA, 31 bp, artificial sequence
  oligonucleotide
gcgaattcatgagatttccttcaatttttac

SEQ ID NO: 27 - DNA, 56 bp, artificial sequence
  oligonucleotide
ggaagcttaaactggcttaccccatctgaagtgttccatagagtaagaagcacctc

SEQ ID NO: 28 - DNA, 44 bp, artificial sequence
  oligonucleotide
ggaagcttaaactggcttaccccatctgaagtgttccatagagt

SEQ ID NO: 29 - DNA, 31 bp, artificial sequence
  oligonucleotide
ccggatccatgagatttccttcaatttttac

SEQ ID NO: 30 - DNA, 33 bp, artificial sequence
  oligonucleotide
gcgaattcagcacctcttttatccaaagatacc

SEQ ID NO: 31 - DNA, 31 bp, artificial sequence
  oligonucleotide
ccatcgatggttctgctagcgccaaaagctc

SEQ ID NO: 32 - DNA, 28 bp, artificial sequence
  oligonucleotide
cagctcgagttagaatagcaggtacgac

SEQ ID NO: 33 - DNA, 27 bp, artificial sequence
  oligonucleotide
cggaattcatggctagcaaaggagaag

SEQ ID NO: 34 - DNA, 29 bp, artificial sequence
  oligonucleotide
ggaagcttttaatcgatgttgtacagttc

SEQ ID NO: 35 - DNA, 20 bp, artificial sequence
  oligonucleotide
gggagcaggggccagtacag

SEQ ID NO: 36 - DNA, 26 bp, artificial sequence
  oligonucleotide
cccaagcttttaccatcaccgtcacc

SEQ ID NO: 37 - DNA, 29 bp, artificial sequence
  oligonucleotide
cccggtaccgtcatgtaattagttatgtc

SEQ ID NO: 38 - DNA, 63 bp, artificial sequence
  oligonucleotide
cgtgcttctggtacatacttgcaatttatacagtgatgaccgctggaccatgattacgccaag

SEQ ID NO: 39 - DNA, 63 bp, artificial sequence
  oligonucleotide
tttagcatggccattgaatgtaacaattatatatatcgcaagcacgattcggtaatctccgag

SEQ ID NO: 40 - DNA, 28 bp, artificial sequence
  oligonucleotide
ccaatgcatggcacagcttgatgcgatc

SEQ ID NO: 41 - DNA, 29 bp, artificial sequence
  oligonucleotide
ccaatgcatgtgtcattggtaaacctgac

SEQ ID NO: 42 - DNA, 31 bp, artificial sequence
  oligonucleotide
gggaagcttttagcttttcattttgatcatc